(19) United States
(12) Patent Application Publication
(10) Pub. No.: US 2026/0284442 A1
(43) Pub. Date: Sep. 24, 2026

(54) A WEARABLE DEVICE FOR APPLYING ULTRASONIC PULSES ON A BLOOD VESSEL OR A TISSUE OF A SUBJECT

(71) Applicant: NOVAPULSE LTD., Or Yehuda (IL)

(72) Inventors: Shaul OZERI, Tel Aviv (IL); Gal ATAROT, Kfar Saba (IL); Shai POLICKER, Raanana (IL); Rann Raphael GREEN, Givatayim (IL)

(21) Appl. No.: 19/477,299

(22) PCT Filed: Apr. 21, 2024
(86) PCT No.: PCT/IL2024/050400

§ 371 Date: Oct. 21, 2025

Related U.S. Application Data
(60) Provisional application No. 63/461,247, filed on Apr. 22, 2023.

(51) Int. Cl. A61N 7/00

(52) U.S. Cl. CPC A61N 7/00; A61N 2007/0091

(57) ABSTRACT

The present disclosure provides a system and a method for non-invasive transmission of ultrasonic (US) pulses towards a target area in a subject. The target area is either a blood vessel or a tissue, in which blood flows. By transmitting US pulses towards the target area, a therapeutic process of the body is enhanced. This therapeutic process is typically associated with the production of nitric oxide molecules, which is induced by insonation. The present disclosure provides a solution that combines a US transducer and one or more sensors. The sensors may sense parameters that indicate either whether the US transducer is in a proper coupling condition and placement over a body part of the subject that allows safe application of US pulses or whether the subject and/or the target area are under allowing conditions for insonation.

### FIELD OF THE INVENTION

The present invention relates generally to a wearable device that delivers therapeutic energy to a patient. More specifically, it relates to an ultrasonic device, adapted to affect vascular endothelium to induce Nitric Oxide (NO) release. Furthermore, the present disclosure relates generally to the treatment of various medical conditions, such as pulmonary hypertension, peripheral arterial disease (PAD), arteriovenous fistula maturation (AVF) and asthma, by enhancing local perfusion to specific target organs.

### BACKGROUND OF THE INVENTION

The emergence of nitric oxide (NO), a reactive, inorganic radical gas as a molecule contributing to important physiological and pathological processes is one of the major biological revelations of recent times.

This molecule is produced under a variety of physiological and pathological conditions by cells mediating vital biological functions. Examples include endothelial cells lining the blood vessels; nitric oxide derived from these cells relaxes smooth muscle and regulates blood pressure and has significant effects on the function of circulating blood cells such as platelets and neutrophils as well as on smooth muscle, both of the blood vessels and also of other organs such as the airways. In the brain and elsewhere nitric oxide serves as a neurotransmitter in non-adrenergic non-cholinergic neurons. In these instances, nitric oxide appears to be produced in small amounts on an intermittent basis in response to various endogenous molecular signals. In the immune system nitric oxide can be synthesized in much larger amounts on a protracted basis. Its production is induced by exogenous or endogenous inflammatory stimuli, notably endotoxin and cytokines elaborated by cells of the host defense system in response to infectious and inflammatory stimuli. This induced production results in prolonged nitric oxide release which contributes both to host defense processes such as the killing of bacteria and viruses as well as pathology associated with acute and chronic inflammation in a wide variety of diseases (Furchgott and Zawadzki 1980; Palmer et al. 1987).

In the field of vascular function, it has been reported that NO generation is stimulated physiologically when the endothelium is exposed to shear stress induced by blood flow and changes in the blood flow (Taso et al. 1995; Uematsu et al. 1995; Ayajiki et al. 1996; Corson et al. 1996; Fleming et al. 1998).

Recent reports have pointed out that ultrasonic application mimics the required shear force to induce vasodilatation, blood flow increases and pH changes in a frequency and amplitude dependent manner, in light of NO generation from the blood vessels (O'Neil R Mason, Brian P Davidson, Paul Sheeran, Matthew Muller, James M Hodovan, Jonathan Sutton, Jeffry Powers, Jonathan R Lindner “Augmentation of Tissue Perfusion in Patients With Peripheral Artery Disease Using Microbubble Cavitation”, JACC Cardiovasc Imaging. 2020 March; 13(3):641-651; J Todd Belcik, Brian H Mott, Aris Xie, Yan Zhao, Sajeevani Kim, Nathan J Lindner, Azzdine Ammi, Joel M Linden, Jonathan R Lindner “Augmentation of limb perfusion and reversal of tissue ischemia produced by ultrasound-mediated microbubble cavitation” Circ. Cardiovasc Imaging. 2015 April; 8(4):e002979; Kiyoshi Iida, Huai Luo, Kohsuke Hagisawa, Takashi Akima, Prediman K Shah, Tasneem Z Naqvi, Robert J Siegel, “Noninvasive low-frequency ultrasound energy causes vasodilation in humans”; J Am Coll Cardiol. 2006 Aug. 1; 48(3):532-7; Matthew A Muller, Koya Ozawa, James Hodovan, Matthew W Hagen, David S H Giraud, Yue Qi, Aris Xie, Theodore R Hobbs, Paul S Sheeran, Jonathan R Lindner, “Treatment of Limb Ischemia with Conducted Effects of Catheter-Based Endovascular Ultrasound”, Ultrasound Med Biol. 2021 August; 47(8):2277-2285).

The vasodilative effect of Nitric Oxide (NO), its positive effect on perfusion and the release of NO from the endothelia (internal layer) of blood vessels is a major natural mechanism to control local and systemic blood pressure, and it is also known that administration of external NO can induce local vasodilation. However, the therapeutic use of systemic NO is limited because of its short half-life (less than 1 sec) and the potential harmful effects that high doses could have on systemic blood pressure.

One of the most prominent possible use of NO induction is in the treatment of peripheral arterial disease (PAD) ischemic tissue. PAD is the narrowing or blockage arteries by atherosclerosis (the build-up of fatty plaque) with or without calcifications.

Another use of NO induction may be for primary pulmonary hypertension. Pulmonary hypertension (PH) or pulmonary arterial hypertension (PAH) is an increase in blood pressure in the pulmonary artery and/or capillaries, together known as the lung vasculature.

NO induction may also be used for treating Raynaud disease. Raynaud's disease is a rare disorder of the blood vessels, usually in the fingers and toes. It causes the blood vessels to narrow under cold stress conditions. Under this condition, blood can't get to the surface of the skin and the affected areas turn white and blue. When the blood flow returns, the skin turns red and throbs or tingles. In severe cases, loss of blood flow can cause sores or tissue death.

Another implementation of NO induction may be for treating arteriovenous fistula (AVF) maturation for patients that have end-stage renal disease (ESRD). In ESRD, kidney function has declined to the point that the kidneys can no longer function on their own. Patients with ESRD must receive hemodialysis or kidney transplantation to survive for more than a few weeks. As a preparation for the hemodialysis, these patients undergo an operation in which an arteriovenous fistula is created in their arm, where a feeding artery such as the radial artery is connected to a venous creating a fistula. It takes about 2-3 months for the fistula to mature. Unfortunately, between 25-50% of the fistulas do not mature.

Thus, as nitric oxide has been proven a potent vasodilator with great therapeutic potential, there is still a long felt need to locally increase availability of nitric oxide at the target tissue (e.g., by utilization of ultrasonic energy) to treat various clinical conditions, such as those disclosed above.

### GENERAL DESCRIPTION

The above disclosed long felt need can be addressed by providing an on-skin device that delivers directed therapeutic energy to a patient, and more specifically, by providing an ultrasonic device adapted to affect, among other things, vascular endothelium to induce nitric oxide (NO) release.

The present invention relates to improving NO release in ischemic tissue by applying ultrasound to the tissue under conditions effective to increase blood flow to the ischemic tissue (e.g., treating ischemic limbs or tissue affected by peripheral arterial disease).

It was found that the application of ultrasound energy to increase NO by an on-skin and/or wearable device requires significant energy focused on the blood vessel; yet more, the continuous application of such energy might result in a reduced efficacy by mechanism of physiological adaptation or other. Thus, to maintain high efficacy, it is beneficial to minimize the application of energy to periods that coincide with natural physiological demand and to limit the duration of such stimulation to an optimum level after which the marginal increase in NO production and vasodilation is diminished.

Therefore, certain embodiments of the present disclosure use at least one sensor embedded within the device, external to the body (e.g., a wearable sensor) or placed elsewhere in the body, to detect the periods in which the patient physiology creates an increased demand for oxygenated blood and enhanced perfusion and thereafter apply energy (e.g., ultrasound) to initiate stimulation of NO production during that time. In some embodiments, the period which is optimal for NO production is tested and the time of stimulation is limited to these periods.

A first configuration of an aspect of the present disclosure provides a wearable device adapted to couple ultrasonic energy through the skin in a patient near, adjacent to, or within at least one vessel or tissue containing flowing blood, comprising means for applying ultrasonic energy to said at least one vessel or tissue containing flowing blood, whereby said means for applying ultrasonic energy, when applied, is adapted to cause a physiologic effect in said at least one vessel or tissue; said device is in communication with at least one remote controller, positioned externally to the patient, adapted to control said means for applying said ultrasonic energy; wherein said device is in communication with at least one sensor adapted to monitor at least one physiological state of the patient such that, upon change thereof, at least one of the following is being performed (a) said ultrasonic energy is delivered to said at least one vessel or tissue, such that an on-demand treatment is provided; (b) at least one treatment parameter of said ultrasonic energy provision to said at least one vessel or tissue is amended, such that an as-needed treatment is provided; (c) said change is notified; (d) any combination thereof.

Another configuration of an aspect of the present disclosure provides non-invasive system for applying ultrasonic (US) pulses, or waves, on or towards a blood vessel or tissue that contains flowing blood of a subject. In other words, the system is configured for providing insonation to a tissue or a blood vessel of a subject.

The system comprises at least one US transducer configured to transmit ultrasonic pulses; a fixation assembly for fixing/securing said at least one US transducer to a body part of the subject that comprises said blood vessel or tissue; at least one sensor configured to monitor at least one physiological parameter or state of the subject and generate sensed data based thereon, for example, a parameter that indicates insufficient blood flow in the blood vessel, which can be monitored by reflections or echoes of US pulses or a parameter that indicates whether the subject sits, walks, or in any other physical position or state, thereby recognizing whether the subject is in a suitable state to receive the US treatment by the system.

The system further comprises at least one processing circuitry, i.e., a controller or a control unit, configured to be in communication with said at least one US transducer and said at least one sensor and configured to receive said sensed data and controllably operate the at least one US transducer in response to said sensed data to transmit US pulse towards said blood vessel or tissue. The controllable operation of the at least one US transducer comprises controlling parameters, such as: intensity, direction, duty cycle, frequency, phase, or any combination thereof.

It is to be noted that any of the below embodiments or objects may refer to any one of the above configurations. Furthermore, any of the below embodiments or objects may apply to any of the above configurations in any combination with other objects or embodiments of any configuration or aspect of the present disclosure.

In some embodiments, the system further comprises a steering arrangement for controllably steering said at least one US transducer towards said blood vessel or tissue.

The term “steering” should be understood as encompassing both physical steering, namely physical change of the position and/or orientation of the transducer such that its main axis is aligned differently in different steering states, and digital steering, namely changing the direction of the US beam such that it reaches a different location, regardless of a physical change of position and/or orientation of the transducer. It is to be noted that the steering of the transducer may include only one of the physical steering and the digital steering and also a combination of the two.

In some embodiments of the system, said steering arrangement comprises steering elements and a chamber filled with flowable material, i.e., liquid or gel. The chamber may comprise flexible walls allowing it to conform with the shape of the skin surface of the subject. At least a portion of said at least one US transducer is disposed within said chamber such that it floats in the flowable material within the chamber. The steering elements are configured to controllably move said at least one US transducer to thereby controllably steer it so as to result in application of US pulse towards a desired location along the blood vessel.

In some embodiments of the system, said flowable material is a non-conducting liquid, such as oil.

In some embodiments of the system, the at least one processing circuitry is configured to execute a periodical steering control loop that comprises controlling the steering arrangement with varying sets of steering parameters and analyzing the sensed data associated with time windows of operation of each set of steering parameters and identifying the optimal set of steering parameters that yield the optimal result of the at least one physiological parameter. In other words, each time window that is analyzed is associated with a specific set of steering parameters, which may include angles with respect to at least two perpendicular planes, wherein each of the planes is normal to the plane defined by the skin of the body part of the subject; wherein upon identification of said optimal set of steering parameters, the at least one processing circuitry is configured to controllably operate the steering arrangement in said optimal set of steering parameters until the next periodical steering control loop. In other words, in each periodical control loop, the steering arrangement is arranged differently in different time windows. For example, in one time window the steering arrangement may be in a first position and in a different time window in a second position. It is to be noted that in each position, the steering parameters may also comprise different operation parameters of the US transducer, such as intensity, phase, or duty cycle. The sensed data may include sensing the pulse wave of blood flow within a blood vessel and once identifying a time window of the optimal blood flow within the blood vessel, wherein the set of steering parameters that are associated with that time window are selected for operation until the next periodical steering control loop.

In some embodiments of the system, said flowable material serves as an impedance couplant, namely the flowable material has a relatively matching impedance to that of the skin tissues.

In some embodiments of the system, said steering elements may include, for example: MEMS based elements, electromagnetically based elements, electrostatic based elements, piezoelectric based elements, magnetic based elements, mechanically based elements, or any other suitable elements.

In some embodiments of the system, said at least one processing circuitry is configured to control the at least one US transducer to apply a check US pulse towards the skin of the subject or towards the blood vessel and detect the reflected echoes resulting from said check pulse. The at least one processing circuitry is configured to analyze said detected reflected echoes to determine whether they satisfy a predetermined condition to thereby determine whether the at least one US transducer is in a suitable contact with a skin portion of the subject. The reflected echoes carry information indicative of the coupling state between the transducer and the skin portion. The application of US pulses from the transducer to the blood vessel is carried out through that skin portion. If said detected reflected echoes do not satisfy said predetermined condition, the at least one processing circuitry is configured to generate an output alert. The alert may be auditory, visual and/or tactile. For example, the output alert may be a sound being heard from the device, an alert presented on a mobile device or a remote controller of the system and/or vibration of mobile device or a remote controller of the system or any other suitable manner for such outputs. Furthermore, when said detected reflected echoes do not satisfy said predetermined condition, the at least one processing circuitry may be configured to disable the operation of the at least one US transducer such that it cannot be activated.

In some embodiments of the system, said predetermined condition is at least one of: a range of time delay from the application of the check pulse and the detection of the reflections, a range of intensities, or a combination thereof.

In some embodiments, the system further comprises first and second electrodes, wherein the at least one US transducer is disposed between said first and second electrodes when the US transducer and the two electrodes contact the skin portion of the subject. Namely, when electric current is applied from a first electrode to the second electrode, the current passes through the skin portion that contacts the US transducer. The at least one processing circuitry is configured to apply electric current from the first electrode to the second electrode so as to measure an electrical parameter. The electrical parameter may be any one of current, impedance, voltage or any combination thereof, The electrical parameter is indicative of a quality of contact between skin portion of the subject and the at least one US transducer. If the quality of contact is below a selected threshold, as identified by the electrical parameter, the at least one processing circuitry is configured to generate an output alert. The alert may be auditory and/or visual. For example, the output alert may be a sound being heard from the device and/or an alert presented on a mobile device or a remote controller of the system, or any other suitable manner for such outputs.

In some embodiments, the system further comprises a first temperature sensor configured to sense a skin temperature in proximity to the at least one US transducer and to generate first skin temperature data based thereon. The at least one processing circuitry is configured to analyze said first skin temperature data and said electrical parameter to determine a subcutaneous temperature of a body area that the US pulse passes through. The at least one processor is configured to analyze a temporal profile of said subcutaneous temperature and to controllably operate the at least one transducer based on identified temporal profile behaviors or signatures of said temporal profile of said subcutaneous temperature. For example, a temporal profile behavior can be an average above a certain temperature threshold over a selected period of time, which requires stopping the application of the US pulses or reducing the intensity or the duty cycle of the US pulses applied on the subject. In another example, the average of the temperature over a period of time may be lower than a certain threshold and the operation of the at least one US transducer is adjusted to provide either more intense pulses or operate in higher duty cycle. It is to be understood that the operation of the at least one US transducer is adjusted based on the temporal profile of the determined subcutaneous temperature.

In some embodiments, there may be multiple electrically conducting electrodes placed on the skin around the ultrasonic transducer. For example, An AC high frequency at about 500 kHz is applied. The high frequency is required for at least two reasons: (1) Being far from the 50-60 Hz noise coupled from the Grid to the skin, therefore allowing simple filtering of the Grid noise out. (2) To couple the current to the tissue through the stratum corneum capacitive dielectric barrier, which is in series with the equivalent tissue impedance, and let the electrode skin-touch impedance to contribute the dominant impedance in the overall equivalent impedance seen by the AC source. The high frequency AC source is connected between the electrodes forming a bipolar or multipolar arrangement (meaning it is not a systemic current flow, but limited to a local flow path between the electrodes. If the electrodes are properly placed on the skin, and a gel, namely a couplant, is also priorly applied on the skin, a closed electrical circuit is formed, and AC current (~1 mA) flow through the electrodes and the skin-surface underlying tissue. If one of the electrodes is not touching, or if they do not completely touch the skin surface, for instance when the device skin touching surface is tilted, the current shall be lower than good-touch current threshold. Therefore, the current between the electrodes if measured, is indicative to the skin-electrode contact quality and therefore indicative of the skin-transducer contact quality.

In addition, the AC source frequency can be swept to increase the sensitivity of the measurement. Sensing the current at multiple frequencies may enable also to estimate the underlying tissue temperature, based on the dependency of the impedance tissue on its temperature.

In some embodiments, the system further comprises at least one force sensor configured to measure the tightening force of the device around the body part caused by the fixation assembly. The at least one processing circuitry is configured to generate a tightening alert if the tightening force is outside of an allowed tightening force range, namely too tight or too loose.

In some embodiments of the system, the force sensor is selected from: force-sensing resistor or strain gauge.

In some embodiments of the system, the body part is selected from a limb (e.g., arm, leg) and the neck.

In some embodiments, the system further comprises an accelerometer configured to sense acceleration of the subject and to generate acceleration data based thereon; the at least one processing circuitry is configured to analyze said acceleration data and determine if the subject is moving. If a movement of the subject is determined, the at least one processing circuitry may be configured to disable the operation of the at least one US transducer, and therefore, to avoid application of US pulses when the subject moves that can lead to non-suitable contact between the at least one transducer and the skin of the subject. When the subject stops moving, the at least one US transducer returns to operate (e.g., automatically). Namely, the operation of the US transducer occurs only in the duration of the detection of the movement of the subject.

In some embodiments, the at least one sensor further comprises a second temperature sensor, that can be the same or different than the first temperature sensor, configured to sense a skin temperature in proximity to the at least one US transducer and to generate second skin temperature data based thereon. The at least one processing circuitry is configured to analyze said second temperature data to determine if the temperature of the skin exceeds allowed temperature threshold and stop the operation of the at least one US transducer upon identification of said exceeding of allowed temperature threshold.

In some embodiments of the system, said response to said sensed data comprises identification of a change of said at least one physiological parameter or identification of a suitable state of the at least one US transducer for applying US pulses, which can be, for example, indication of a suitable contact of the at least one US transducer with the body part; wherein upon identification of said change, said suitable state or a combination thereof, at least one of the following is being performed: (a) said US pulse is transmitted to said at least one vessel or tissue, such that an on-demand treatment is provided; (b) at least one treatment parameter of said US pulse transmission to said at least one vessel or tissue is changed, such that an as-needed treatment is provided; (c) outputting a notification output of said change; (d) any combination thereof.

In some embodiments of the system, said at least one sensor comprises acoustic sensor that can be part of the US transducer or independent sensor, configured to detect acoustic signal from skin portion of the subject and generate acoustic sensed data, said acoustic sensed data being indicative of blood flow in said blood vessel or tissue. The sensed data may comprise said acoustic sensed data, namely the US transducer is operated based on the blood flow profile that is interpreted from the acoustic sensed data.

In some embodiments of the system, the at least one sensor is configured to sense at least one pulse wave characteristic and determine heart rate of the subject from said at least one pulse wave characteristic. Analysis of said at least one pulse wave characteristic facilitates alignment of the position of said US transducer relatively to said at least one blood vessel or tissue. Namely, the pulse wave characteristic may affect the operation of the steering assembly such that the steering elements move the US transducer in response to the analysis of the pulse wave characteristic; wherein said facilitating comprises either outputting an indication of desired alignment of the US transducer or direct control of the steering assembly or, specifically, steering elements.

In some embodiments of the system, said at least one sensor is an ultrasonic sensor configured to sense ultrasonic signals. The ultrasonic sensor may be constituted by the at least one US transducer. Namely, the at least one US transducer may be configured for application of US pulses and sensing US signals.

In some embodiments of the system, the at least one sensor is configured to sense the US doppler parameter of the applied US pulses. The US doppler parameter is indicative of the blood flow in the tissue or the blood vessel. Namely, the US doppler parameter is, indirectly, the at least one physiological parameter.

In some embodiments of the system, the at least one sensor comprises an ECG sensor and the US transducer. Namely, in this embodiment, the system is configured to sense an ECG signal by the ECG sensor and also acoustic signals by the at least one US transducer. The acoustic signals are generated by blood pulse when flowing through the blood vessel or the tissue. The at least one processing circuitry is configured to determine time delays between the ECG signal and the onset of the blood pulse wave sensed by the at least one US transducer. The time delay variation profile over time is indicative of the change of blood flow in the tissue or blood vessel. The at least one processing circuitry is configured to controllably operate the at least one US transducer based on said variation profile. In other words, the variation profile provides indication whether the insonation is efficient or not and the parameters of the insonation are controlled based on the identified variation profile.

In some embodiments of the system, the at least one sensor is the at least one US transducer and is configured to sense acoustic signals that are generated by blood pulses when flowing through the blood vessel or the tissue. The at least one processing circuitry is configured to determine a variation profile of said acoustic signals and operate the at least one US transducer to apply pulses in response to said variation profile. In other words, the change of the shape of the acoustic pulse wave indicates the effectivity of the insonation and the operation of the at least one US transducer is performed in response to the identification of signatures change over time of the shape of these acoustic signals.

In some embodiments of the system, the at least one sensor is the at least one US transducer and it is configured to sense acoustic signals that are generated by blood pulses when flowing through the blood vessel or the tissue. The at least one processing circuitry is configured to synchronize the application of US pulses by the at least one processing circuitry with the onset or the appearance of the acoustic pulse wave. This may be beneficial, for instance, to augment the shear effect.

In some embodiments of the system, the at least one sensor comprises one or more of a temperature sensor and an IR sensor for measuring a skin perfusion parameter indicative of the skin perfusion in a tissue associated with the blood vessel or the tissue. The association can be that this skin portion is downstream the blood flow path to the tissue or blood vessel that are treated. This can be by a direct temperature measurement or by an IR measurement of radiation from the skin portion. The skin perfusion is indicative of the microcirculatory blood flow that depends on the feeding artery blood flow, and the artery blood flow depends on the insonation. Therefore, the at least one US transducer is operated in response for the sensing of said skin perfusion parameter. It is to be noted that the skin perfusion parameter may be also determined by an indirect measurement of Arterial Pulse Oximeter Pleth (Plethysmograph).

In some embodiments, the system comprises an array of US sensors/transducers, wherein the at least one processing circuitry is configured to controllably operate the array of US transducers for steering the US pulse to a desired direction.

In some embodiments of the system, the array of US sensors/transducers operates as a phased array for said steering.

In some embodiments of the system, the at least one processing circuitry is configured to execute a periodical control loop that comprises controllably operating the array of US transducers with varying sets of operation parameters and analyzing the sensed data associated with time windows of operation of each set of operation parameters and identifying the optimal set of operation parameters that yield the optimal result of the at least one physiological parameter; wherein upon identification of said optimal set of operation parameters, the at least one processing circuitry is configured to controllably operate the plurality of US transducers in said optimal set of operation parameters until the next periodical control loop. In other words, in each periodical control loop, the array of US transducers operate differently in different time windows. For example, in one time window only some of the US transducers may operate while others may not, and in a different time window, other combination of US transducers operate. Also, in each operation of combination of US transducers, there may be varying operation parameters of the US transducers over time to identify the optimal operation parameters of the specific combination of US transducers. The sensed data may include sensing the pulse wave of blood flow within a blood vessel and once identifying a time window of the optimal blood flow within the blood vessel, wherein the set of operation parameters that are associated with that time window are selected for operation until the next periodical control loop.

In some embodiments of the system, the varying sets of operation parameters comprise one or more of: varying intensity, phase or duty cycle of each US transducer of the array of transducers.

In some embodiments, upon change of said at least one physiological parameter/state, said device is self-activated.

In some embodiments, upon change of said at least one physiological parameter/state, the user (e.g., patient, caregiver) may activate said ultrasonic energy or amends said at least one treatment parameter.

In some embodiments, the at least one sensor configured to monitor at least one physiological parameter/state of the subject may be, for example: accelerometer, acoustic impedance measurement, electrical impedance measurement, photoplethysmography sensor, PPG sensor, pH sensor, ultrasound sensor, echocardiogram, ultrasound echo, temperature meter, body core temperature, heart pulse rate, blood pulse wave properties, glucose sensor, any sensor indicating a change in cardiac output, any sensor indicating blood pressure, any sensor indicating initiation of a dialysis session, any sensor associated with a dialysis machine and any combination thereof.

In some embodiments, the at least one sensor may be configured to sense/monitor, for example: movement of said patient, impedance, PPG signal, pH, acoustic signal, pressure, temperature, heart rate, pulse wave properties, glucose level, blood pressure, and any combination thereof.

In some embodiments, the at least one sensor may be integrated within the device, a sensor being worn by the patient, a remote sensor outside the patient's body, a sensor integrated within the at least one controller, and any combination thereof.

In some embodiments, the monitored physiological parameter/state or change thereof is one or more of, for example: position of the patient, engagement of the patient in physical activity, decrease in NO levels in said at least one vessel or tissue, tissue perfusion, initiation of a physical activity, change in at least one parameter associated with said physical activity, the position of the patient relative to the ground, changes in said position of the patient relative to the ground, application of at least one medical treatment to said patient, changes in application of at least one medical treatment to said patient and any combination thereof.

In some embodiments, the at least one treatment parameter is, for example: a phase, an operating center frequency, power, intensity, operating amplitude, timing, duration, orientation and any combination thereof of said ultrasonic energy. in some embodiments, upon change of said at least one physiological state, at least one notification is sent to said patient or any caregiver thereof.

In some embodiments, said device may be in communication with at least one battery. The at least one battery may be positioned externally to the patient. The at least one battery may be configured to be wirelessly charged.

In some embodiments, the physiologic effect is, for example: vasodilation, an increase in local nitric oxide, enhanced nitric oxide release from the vascular endothelium; prolonged local nitric oxide effects, an alteration in the function of erythrocytes, a modification in oxygen release from hemoglobin, blood temperature increase, modification in pH of blood, modulation in the immune response of blood leucocytes, modulation of the coagulation and/or thrombocyte function, modification in the function of heme catalyst enzymes in the blood, improved bioavailability of medication, improved efficiency of a hemodialysis session artery dilation, increased blood perfusion and any combination thereof.

In some embodiments, the ultrasonic energy is provided in a center frequency in the range from about 10 kHz to about 10 MHz.

In some embodiments, the ultrasonic energy is provided by at least one of, for example: at least one piezoelectric transducer which generates ultrasound energy, at least one passive ferromagnetic element, at least one capacitive micromachined ultrasonic transducer, CMUT, concave transducer, convex transducer and any combination thereof.

In some embodiments, said piezoelectric transducer is made of at least one of, for example: lead zirconate titanate, lead magnesium niobate-lead titanate, Hard PZT, composite and any combination thereof.

In some embodiments, said piezoelectric transducer is shaded by means of apodization, for instance Gaussian apodization. Said Gaussian apodization may be provided by material polarization.

In some embodiments, said piezoelectric transducers generates an ultrasonic energy pulse with more than one center frequency.

In some embodiments, at least one of said controllers is adapted to charge said device.

In some embodiments, said at least one sensor is adapted to sense at least one pulse wave characteristic.

In some embodiments, said at least one sensor is adapted to sense at least one pulse wave characteristic reflected from said device by means of, for example, acoustic sensing, echo and any combination thereof.

In some embodiments, the device is adapted to sense heart rate from said at least one pulse wave characteristic.

In some embodiments, analysis of said at least one pulse wave characteristic facilitates alignment of the position and/or orientation of said device relatively to said at least one vessel or tissue containing flowing blood.

In some embodiments, said alignment is indicated to either the patient or a caregiver thereof. Said alignment may be indicated by at least one indication means, for example: audio means, visual means, tactile means, vibration means and any combination thereof.

In some embodiments, analysis of said at least one pulse wave characteristic indicates said causing of said physiologic effect.

In some embodiments, the ultrasonic energy is provided by an array of piezoelectric transducers, each of which generates ultrasound energy. Said array of piezoelectric transducers may be a phased array.

In some embodiments, activation of at least one of said transducers according to a predetermined protocol results in finetuning of said ultrasonic energy direction relative to said at least one vessel so as to align said device thereto.

In some embodiments, said ultrasonic energy may be provided in a continuous manner, or alternatively, in pulses.

In some embodiments, at least one of said controllers is adapted to collect data from said at least one sensor and perform at least one of, for example: (a) monitor said data; (b) amend at least one treatment parameter of the treatment protocol; (c) maintain the treatment provided to said patient as is; and any combination thereof.

In some embodiments, at least one of said controllers is a wearable by said patient. Said controller may be integrated in, for example, a sock, shoe, glove, sleeve, clothes, hats and any combination thereof.

In some embodiments, at least one of said controllers is integrated in said patient's environment.

In some embodiments, at least one of said controllers is in communication with a processing unit, such as, CPU, smartphone, and any combination thereof.

In some embodiments, the disclosed devices and systems may be used for treating various clinical conditions, such as: pulmonary artery denervation, pulmonary hypertension, ischemic tissues, PAD, CLI, pulmonary artery hypertension, arteriovenous fistula (AVF) maturation, Raynaud disease, severe asthma patients, improve blood flow to the brain during stroke, increase blood flow to the penis to maintain an erection, enhancement of bioavailability of medications, enhancement of local chemotherapy absorption into a solid tumor by enhancing flow of specific arteries feeding the tumor and any combination thereof.

In some embodiments, said ischemic tissue may be in, for example, the upper limbs, lower limbs, arms, legs and any combination thereof.

In some embodiments, the purpose of applying said ultrasonic energy is to increase blood flow to the target tissue.

In some embodiments, said ultrasonic energy is provided in at least two different center frequencies. The first center frequency may be in a range from about 10 kHz to about 10 MHz. The second center frequency may be in a range from about 100 kHz to about 10 MHz.

In some embodiments, said device comprises at least one piezoelectric transducer adapted to generate said ultrasonic energy in one of said at least two different center frequencies.

In some embodiments, said device additionally comprises at least one electro-magnetic acoustic transducer mechanically coupled to said at least one piezoelectric transducer adapted to generate said ultrasonic energy in one of said at least two different center frequencies. The at least one electro-magnetic acoustic transducer may be at least one ferromagnetic sheet.

In some embodiments, said device comprises at least one processor configured to control at least one operation/treatment parameter, such as: a phase, an operating center frequency, power, intensity, operating amplitude, timing, duration, orientation, and any combination thereof of said ultrasonic energy.

In some embodiments, said at least one processor is in communication with said at least one sensor.

In some embodiments, said at least one processor is configures to collect data from said at least one sensor and perform at least one action, such as: (a) monitor said data; (b) amend the treatment provided to said patient; (c) maintain the treatment provided to said patient as is; and any combination thereof.

Another aspect of the present invention provides a first method definition, The first method definition provides a method of treating a patient, comprising steps of:
a. providing at least one device adapted to be placed on skin in a patient near, adjacent to, or within at least one vessel or tissue containing flowing blood, comprising means for applying ultrasonic energy to said at least one vessel or tissue containing flowing blood, whereby said means for applying ultrasonic energy, when applied, is adapted to cause a physiologic effect in said at least one vessel or tissue; said device is in communication with at least one remote controller, positioned externally to the patient, adapted to control said means for applying said ultrasonic energy;
b. placing said at least one device adjacent to, or within at least one vessel or tissue containing flowing blood;
c. communicating said device with at least one sensor adapted to monitor at least one physiological state of the patient such that, upon change thereof, at least one of the following is being performed (a) said ultrasonic energy is delivered to said at least one vessel or tissue, such that an on-demand treatment is provided; (b) at least one treatment parameter of said ultrasonic energy provision to said at least one vessel or tissue is amended, such that an as-needed treatment is provided; (c) said change is notified; (d) any combination thereof; thereby treating said patient.

Yet another method definition of this aspect provides a method for non-invasively applying ultrasonic (US) pulses, or waves, namely performing insonation, on or towards a blood vessel or tissue of a subject. The method comprises: fixing or securing at least one ultrasonic (US) transducer to a body part of the subject that comprises said blood vessel or tissue; monitoring at least one physiological parameter and generating sensed data based thereon; operating said at least one US transducer in response to said sensed data to transmit US pulse towards said blood vessel or tissue. Said operating of the at least one US transducer comprises controlling parameters selected from: intensity, direction, duty cycle, frequency, phase, or any combination thereof.

In some embodiments of the method, said operating comprises steering said at least one US transducer towards said blood vessel or tissue.

In some embodiments, the method further comprises disposing at least a portion of said at least one US transducer in a chamber filled with flowable material, i.e., liquid or gel, and controllably moving said at least one US transducer to thereby controllably steer it.

In some embodiments of the method, said controllably moving is performed by steering elements, wherein said steering elements are selected from any one of: MEMS based elements, electromagnetically based elements, magnetic based elements, or mechanically based elements.

In some embodiments of the method, said flowable material is a non-conducting liquid (such as oil).

In some embodiments of the method, said flowable material serves as an impedance couplant, namely, the flowable material has a relatively matching impedance to that of the skin tissues.

In some embodiments, the method further comprises executing a periodical steering control loop that comprises steering said at least one US transducer with varying sets of steering parameters and analyzing the sensed data associated with time windows of operation of each set of steering parameters and identifying the optimal set of steering parameters that yield the optimal result of the at least one physiological parameter; wherein upon identification of said optimal set of steering parameters, the method further comprises steering said at least one US transducer in said optimal set of steering parameters until the next periodical steering control loop.

In some embodiments, the method further comprises applying a check US pulse towards the skin of the subject or towards the blood vessel with the at least one US transducer and detecting the reflected echoes resulting from said check pulse; analyzing said detected reflected echoes to determine whether they satisfy a predetermined condition to thereby determine whether the at least one US transducer is in a suitable contact with a skin portion of the subject. The application of US pulses from the transducer to the blood vessel are carried out through that skin portion; wherein if said detected reflected echoes do not satisfy said predetermined condition, method further comprises generating an output alert. The output alert can be a sound being heard from the device, an alert presented on a mobile device or a remote controller of the system or any other suitable manner for such outputs. Furthermore, when said detected reflected echoes do not satisfy said predetermined condition, the method further comprises disabling the operation of the at least one US transducer such that it cannot be activated.

In some embodiments of the method, said predetermined condition is at least one of: a range of time delay from the application of the check pulse and the detection of the reflections, a range of intensities, or a combination thereof.

In some embodiments, the method further comprises attaching first and second electrodes to a skin portion of the subject, wherein the at least one US transducer is disposed between said first and second electrodes when the US transducer and the two electrodes contact the skin portion of the subject. Namely, when electric current is applied from a first electrode to the second electrode, the current passes through the skin portion that contacts the US transducer; wherein the method further comprises applying electric current from the first electrode to the second electrode so as to measure an electrical parameter, the electrical parameter is selected from any one of: current, impedance, voltage or any combination thereof. The electrical impedance is indicative of a quality of contact between the skin portion of the subject and the at least one US transducer; wherein if the quality of contact is below a selected threshold, the method comprises generating an output alert. The output alert can be a sound being heard from the device, an alert presented on a mobile device or a remote controller of the system or any other suitable manner for such outputs.

In some embodiments, wherein the at least one physiological parameter comprises a skin temperature in proximity to the at least one US transducer generating first skin temperature data based thereon; wherein the method further comprises analyzing said first skin temperature data and said electrical impedance to determine a subcutaneous temperature. The method further comprises analyzing a temporal profile of said subcutaneous temperature and controllably operating the at least one transducer based on identified temporal profile behaviors of said temporal profile of said subcutaneous temperature.

In some embodiments, the method further comprises measuring tightening force of the device around the body part caused by said fixing; wherein the method comprises generating a tightening alert if the tightening force is outside of an allowed tightening force range, namely too tight or too loose.

In some embodiments of the method, said measuring is performed by at least one force sensor selected from: force-sensing resistor or strain gauge.

In some embodiments of the method, the body part is selected from a limb and neck.

In some embodiments, the method further comprises sensing acceleration of the subject and generating acceleration data based thereon; wherein the method comprises analyzing said acceleration data and determining if the subject is moving; if a movement of the subject is determined, the method comprises disabling the operation of the at least one US transducer, and therefore, avoiding application of US pulses when the subject moves that can lead to non-suitable contact between the at least one transducer and the skin of the subject. When the subject stops moving, the at least one US transducer returns to operate. Namely, the operation of the US transducer occurs only in the duration of the detection of the movement of the subject.

In some embodiments, the method further comprises sensing a skin temperature in proximity to the at least one US transducer and generating second skin temperature data based thereon; wherein the method comprises analyzing said second temperature data to determine if the temperature of the skin exceeds allowed temperature threshold and stopping the operation of the at least one US transducer upon identification of said exceeding of allowed temperature threshold.

In some embodiments of the method, said response to said sensed data comprises identification of a change of said at least one physiological parameter; wherein upon identification of said change or identification of a suitable state of the at least one US transducer for applying US pulses, which can be, for example, indication of a suitable contact of the at least one US transducer with the body part; wherein upon identification of said change, said suitable state or a combination thereof, at least one of the following is being performed: (a) said US pulse is transmitted to said at least one vessel or tissue, such that an on-demand treatment is provided; (b) at least one treatment parameter of said US pulse transmission to said at least one vessel or tissue is changed, such that an as-needed treatment is provided; (c) outputting a notification output of said change; (d) any combination thereof.

In some embodiments, the method further comprises detecting acoustic signal from skin portion of the subject and generating acoustic sensed data, said acoustic sensed data being indicative of blood flow in said blood vessel or tissue; wherein said sensed data comprises said acoustic sensed data. Namely, the US transducer is operated based on the blood flow profile that is interpreted from the acoustic sensed data.

In some embodiments of the method, the at least one physiological parameter is monitored by the said at least at least one US transducer. Namely, the at least one US transducer is configured application of US pulses and sensing US signals.

In some embodiments of the method, the at least one physiological parameter is US doppler parameter of the applied US pulses by the at least one US transducer.

In some embodiments of the method, the at least one physiological parameter comprises an ECG signal and acoustic signals. The acoustic signals are generated by blood pulse when flowing through the blood vessel or the tissue. The method further comprises determining time delays between the ECG signal and the onset of the blood pulse wave sensed in said acoustic signals. The time delay variation profile over time is indicative of the change of blood flow in the tissue or blood vessel. The method further comprises operating the at least one US transducer based on said variation profile.

In some embodiments of the method, the at least one physiological parameter comprises acoustic signals that are generated by blood pulse when flowing through the blood vessel or the tissue. The method further comprises determining variation profile of said acoustic signals and operating the at least one US transducer to apply pulses in response to said variation profile.

In some embodiments of the method, the at least one physiological parameter comprises acoustic signals that are generated by blood pulse when flowing through the blood vessel or the tissue. The method further comprises synchronizing the application of US pulses by the at least one processing circuitry with the onset or the appearance of the acoustic pulse wave.

In some embodiments of the method, the at least one physiological parameter comprises a skin perfusion parameter indicative of the skin perfusion in a tissue associated with the blood vessel or the tissue. The skin perfusion parameter can be obtained by an IR or temperature measurement. The skin perfusion is indicative of the microcirculatory blood flow that depends on the feeding artery blood flow, and the artery blood flow depends on the insonation. Therefore, the at least one US transducer is operated in response for the sensing of said skin perfusion parameter. It is to be noted that the skin perfusion parameter may be also determined by an indirect measurement of Arterial Pulse Oximeter Pleth (Plethysmograph).

In some embodiments of the method, said at least one US transducer comprises an array of US transducers, wherein the method further comprises controllably operating the array of US transducers for steering the US pulse to a desired direction.

In some embodiments of the method, the array of US transducers operates as a phased array for said steering.

In some embodiments, the method further comprises executing a periodical control loop that comprises controllably operating the array of US transducers with varying sets of operation parameters and analyzing the sensed data associated with time windows of operation of each set of operation parameters and identifying the optimal set of operation parameters that yield the optimal result of the at least one physiological parameter; wherein upon identification of said optimal set of operation parameters, the method further comprises controllably operating the plurality of US transducers in said optimal set of operation parameters until the next periodical control loop.

In some embodiments of the method, the blood vessel is arteriovenous fistula (AVF) and the method comprises insonating the AVF to enhance its maturation.

In some embodiments, the method comprises orienting at least two devices at the same position relative to said at least one vessel or tissue containing flowing blood.

In some embodiments, the method comprises orienting at least two devices at a substantially different position relative to said at least one vessel or tissue containing flowing blood.

In some embodiments, the method comprises enabling focusing said ultrasonic energy on said at least one vessel or tissue by means of said positioning of said at least two devices.

In some embodiments, the method comprises enabling communication between said at least two devices.

In some system embodiments, at least two of a plurality of devices are in communication with each other another.

Yet another aspect of the present disclosure provides a system for treatment of a patient's blood vessel. The system comprises: a blood vessel sleeve configured to be fitted around the outside of at least a portion of a blood vessel, the sleeve comprises at least one ultrasonic (US) transducer configured to provide ultrasonic energy to said at least a portion of a blood vessel, and a first sensor configured to monitor at least one physiological parameter of the patient and generate sensed data based thereon; and a remote controller disposed external to the patient and is communicable with said at least one ultrasonic transducer and said first sensor for controllably operating the at least one ultrasonic transducer and receiving sensed data from the sensor.

In some embodiments, the system further comprises an energy transmitting unit intended to place external to the patient and an energy receiving unit coupled to or integral with the blood vessel sleeve; wherein the energy transmitting unit is configured to transmit energy externally to the patient towards the energy receiving unit for energizing the US transducer, and therefore allowing it to operate at a desired operation mode according to the controlled operation by the remote controller.

In some embodiments of the system, the remote controller comprises said energy transmitting unit.

In some embodiments of the system, the energy receiving unit is a first inductor and the energy transmitting unit is a second inductor.

In some embodiments of the system, the energy receiving unit is coupled to the blood vessel sleeve by a pig-tail connection.

Yet another aspect of the present disclosure provides a method for treatment of a patient's blood vessel. The method comprises: fitting a blood vessel sleeve around the outside of at least a portion of a blood vessel, the sleeve comprises at least one ultrasonic (US) transducer configured to provide ultrasonic energy to said at least a portion of a blood vessel, and a first sensor configured to monitor at least one physiological parameter of the patient and generate sensed data based thereon; and controllably operating the at least one ultrasonic transducer and receiving sensed data from the sensor.

In some embodiments of the method, said blood vessel is a bypass blood vessel bypassing an artery narrowing portion.

Various other objects, aspects and advantages of the present inventive disclosure can be obtained from a study of the specification, the drawings, and the appended claims.

### EMBODIMENTS

The following are optional embodiments and combinations thereof in accordance with aspects of the present disclosure:
1. A non-invasive system for applying ultrasonic (US) pulses on or towards a blood vessel or tissue that contains flowing blood of a subject, comprising:
   at least one US transducer configured to transmit ultrasonic pulses;
   a fixation assembly configured for fixing or securing said at least one US transducer to a body part of the subject that comprises said blood vessel or tissue;
   at least one sensor configured to monitor at least one physiological parameter or state of the subject and generate sensed data based thereon;
   at least one processing circuitry configured to be in data communication with said at least one US transducer and with said at least one sensor and configured to receive said sensed data and controllably operate the at least one US transducer in response to said sensed data to transmit US pulse towards said blood vessel or tissue.
2. The system of embodiment 1, comprising steering arrangement configured to controllably steer said at least one US transducer towards said blood vessel or tissue.
3. The system of embodiment 2, wherein said steering arrangement comprises steering elements and a chamber filled with flowable material, wherein at least a portion of said at least one US transducer is disposed within said chamber, wherein said steering elements are configured to controllably move said at least one US transducer to thereby controllably steer it.
4. The system of embodiment 3, wherein said flowable material is a non-conducting liquid.
5. The system of either one of embodiments 3 or 4, wherein said flowable material serves as an impedance couplant.
6. The system of any one of embodiments 3-5, wherein said steering elements are selected from any one of: MEMS based elements, electromagnetically based elements, electrostatic based elements, piezoelectric based elements, magnetic based elements, or mechanically based elements.
7. The system of any one of embodiments 2-6, wherein the at least one processing circuitry is configured to execute a periodical steering control loop that comprises controlling the steering arrangement with varying sets of steering parameters and analyzing the sensed data associated with time windows of operation of each set of steering parameters and identifying the optimal set of steering parameters that yield the optimal result of the at least one physiological parameter; wherein upon identification of said optimal set of steering parameters, the at least one processing circuitry is configured to controllably operate the steering arrangement in said optimal set of steering parameters until the next periodical steering control loop.
8. The system of any one of embodiments 1-7, wherein said at least one processing circuitry is configured to control the at least one US transducer to apply a check US pulse and detect the reflected echoes resulting from said check pulse; wherein the at least one processing circuitry is configured to analyze said detected reflected echoes to determine whether they satisfy a predetermined condition to thereby determine whether the at least one US transducer is in a suitable contact with a skin portion of the subject; wherein if said detected reflected echoes do not satisfy said predetermined condition, the at least one processing circuitry is configured to generate an output alert.
9. The system of embodiment 8, wherein said predetermined condition is at least one of: a range of time delay from the application of the check pulse and the detection of the reflections, a range of intensities, or a combination thereof.
10. The system of any one of embodiments 1-9, comprising first and second electrodes, wherein the at least one US transducer is disposed between said first and second electrodes; wherein the at least one processing circuitry is configured to apply electric current from the first electrode to the second electrode so as to measure an electrical parameter selected from impedance, current and voltage; wherein the electrical parameter being indicative of a quality of contact between skin portion of the subject and the at least one US transducer; wherein if the quality of contact is below a selected threshold, the at least one processing circuitry is configured to generate an output alert.
11. The system of embodiment 10, wherein said at least one sensor comprises a first temperature sensor configured to sense a skin temperature in proximity to the at least one US transducer and to generate first skin temperature data based thereon; wherein the at least one processing circuitry is configured to analyze said first skin temperature data and said electrical parameter to determine a subcutaneous temperature; wherein the at least one processor is configured to analyze a temporal profile of said subcutaneous temperature and to controllably operate the at least one transducer based on identified temporal profile behaviors of said temporal profile of said subcutaneous temperature.
12. The system of any one of embodiments 1-11, wherein said at least one sensor comprises at least one force sensor configured to measure the tightening force of the device around the body part caused by the fixation assembly; wherein the at least one processing circuitry is configured to generate a tightening alert if the tightening force is outside of an allowed tightening force range.
13. The system of embodiment 12, wherein said at least one force sensor is selected from: force-sensing resistor or strain gauge.
14. The system of any one of embodiments 1-13, wherein said at least one sensor comprises an accelerometer configured to sense acceleration of the subject and to generate acceleration data based thereon; wherein the at least one processing circuitry is configured to analyze said acceleration data and determine if the subject is moving; wherein if a movement of the subject is determined, the at least one processing circuitry is configured to disable the operation of the at least one US transducer.
15. The system of any one of embodiments 1-14, wherein said at least one sensor comprises a second temperature sensor configured to sense a skin temperature in proximity to the at least one US transducer and to generate second skin temperature data based thereon; wherein the at least one processing circuitry is configured to analyze said second temperature data to determine if the temperature of the skin exceeds an allowed temperature threshold and stop the operation of the at least one US transducer upon identification of said exceeding of said allowed temperature threshold.
16. The system of any one of embodiments 1-15, wherein said at least one sensor comprises an acoustic sensor configured to detect acoustic signals from a skin portion of the subject and generate acoustic sensed data, said acoustic sensed data being indicative of blood flow in said blood vessel or tissue; wherein said sensed data comprises said acoustic sensed data.
17. The system of any one of embodiments 1-16, wherein said response to said sensed data comprises identification of a change of said at least one physiological parameter; wherein upon identification of said change at least one of the following is performed: (a) said US pulse is transmitted to said at least one vessel or tissue, such that an on-demand treatment is provided; (b) at least one treatment parameter of said US pulse transmission to said at least one vessel or tissue is changed, such that an as-needed treatment is provided; (c) outputting a notification of said change; (d) any combination thereof.
18. The system of any one of embodiments 1-17, wherein said at least one sensor is selected from a group consisting of accelerometer, impedance measurement, photoplethysmography sensor, PPG sensor, pH sensor, ultrasound sensor, echocardiogram, ultrasound echo, hydrophone, temperature meter, body core temperature, heart pulse rate, pulse wave properties, glucose sensor, any sensor indicating a change in cardiac output, any sensor indicating blood pressure, any sensor indicating initiation of a dialysis session, any sensor associated with dialysis machine and any combination thereof.
19. The system of 18, wherein said sensor is adapted to sense at least one parameter selected from a group consisting of movement of said patient, impedance, PPG signal, acoustic signal, pressure, temperature, heart rate, pulse wave properties, glucose level, blood pressure, and any combination thereof.
20. The system of any one of embodiments 1-19, wherein said physiological parameter or state or change thereof is selected from a group consisting of position of the patient, engagement in physical activity, decrease in NO levels in said at least one vessel or tissue, tissue perfusion, initiation of a physical activity, change in at least one parameter associated with said physical activity, the position of the patient relative to the ground, changes in said position of the patient relative to the ground, application of at least one medical treatment to said patient, changes in application of at least one medical treatment to said patient and any combination thereof.
21. The system of any one of embodiments 1-20, wherein said controllably operate the at least one US transducer comprises controlling at least one treatment parameter selected from a group consisting of a phase, an operating frequency, power, intensity, operating amplitude, timing, duration, orientation toward a blood vessel and any combination thereof of said ultrasonic energy.
22. The system of any one of embodiments 1-21, wherein said controllably operate the at least one US transducer results in a physiologic effect selected from the group consisting of: vasodilation; an increase in local nitric oxide; enhance nitric oxide release from the vascular endothelium; prolong local nitric oxide effects; an alteration in the function of erythrocytes; a modification in oxygen release from hemoglobin, blood temperature increase; modification in pH of blood; modulation in the immune response of blood leucocytes; modulation of the coagulation and/or thrombocyte function; modification in the function of heme catalyst enzymes in the blood, improved bioavailability of medication, improved efficiency of a hemodialysis session artery dilation, increased blood perfusion and combinations thereof
23. The system of any one of embodiments 1-22, wherein the at least one sensor is configured to sense at least one pulse wave characteristic and determine heart rate of the subject from said at least one pulse wave characteristic; wherein analysis of said at least one pulse wave characteristic facilitates alignment of the position of said US transducer relatively to said at least one blood vessel or tissue.
24. The system of any one of embodiments 1-23, wherein said at least one sensor is an ultrasonic sensor configured to sense ultrasonic signals, wherein the ultrasonic sensor is constituted by the at least one US transducer.
25. The system of any one of embodiments 1-24, comprising an array of US transducers, wherein the at least one processing circuitry is configured to controllably operate the array of US transducers for steering the US pulse to a desired direction.
26. The system of embodiment 25, wherein the array of US transducers operates as a phased array for said steering.
27. The system of embodiment 25 or 26, wherein the at least one processing circuitry is configured to execute a periodical control loop that comprises controllably operating the array of US transducers with varying sets of operation parameters and analyzing the sensed data associated with time windows of operation of each set of operation parameters and identifying the optimal set of operation parameters that yield the optimal result of the at least one physiological parameter; wherein upon identification of said optimal set of operation parameters, the at least one processing circuitry is configured to controllably operate the plurality of US transducers in said optimal set of operation parameters until the next periodical control loop.
28. The system of embodiment 27, wherein the varying sets of operation parameters comprise varying intensity, phase or duty cycle of each US transducer of the array of transducers.
29. A method for non-invasively applying ultrasonic (US) pulses on or towards a blood vessel or tissue of a subject, comprising:
   fixing at least one ultrasonic (US) transducer to a body part of the subject that comprises said blood vessel or tissue;
   monitoring at least one physiological parameter and generating sensed data based thereon; and
   operating said at least one US transducer in response to said sensed data to transmit US pulse towards said blood vessel or tissue.
30. The method of embodiment 29, wherein said operating comprises steering said at least one US transducer towards said blood vessel or tissue.
31. The method of embodiment 30, comprising disposing at least a portion of said at least one US transducer in a chamber filled with flowable material and controllably moving said at least one US transducer to thereby controllably steer it.
32. The method of embodiment 31, wherein said controllably moving is performed by steering elements, wherein said steering elements are selected from any one of: MEMS based elements, electromagnetically based elements, magnetic based elements, or mechanically based elements.
33. The method of either one of embodiments 31 or 32, wherein said flowable material is a non-conducting liquid.
34. The method of any one of embodiments 29-33, wherein said flowable material serves as an impedance couplant.
35. The method of any one of embodiments 29-34, comprising executing a periodical steering control loop that comprises steering said at least one US transducer with varying sets of steering parameters and analyzing the sensed data associated with time windows of operation of each set of steering parameters and identifying the optimal set of steering parameters that yield the optimal result of the at least one physiological parameter; wherein upon identification of said optimal set of steering parameters, the method further comprises steering said at least one US transducer in said optimal set of steering parameters until the next periodical steering control loop.
36. The method of any one of embodiments 29-35, comprising:
   applying a check US pulse with the at least one US transducer;
   detecting the reflected echoes resulting from said check pulse;
   analyzing said detected reflected echoes to determine whether they satisfy a predetermined condition to thereby determine whether the at least one US transducer is in a suitable contact with a skin portion of the subject; and
   if said detected reflected echoes do not satisfy said predetermined condition, generating an output alert.
37. The method of embodiment 36, wherein said predetermined condition is at least one of: a range of time delay from the application of the check pulse and the detection of the reflections, a range of intensities, or a combination thereof.
38. The method of any one of embodiments 29-37, comprising:
   attaching first and second electrodes to a skin portion of the subject, wherein the at least one US transducer is disposed between said first and second electrodes;
   applying electric current from the first electrode to the second electrode so as to measure an electrical parameter selected from current, impedance or voltage; wherein the electrical parameter indicative of a quality of contact between the skin portion of the subject and the at least one US transducer; and
if the quality of contact is below a selected threshold, generating an output alert.
39. The method of embodiment 38, comprising:
   sensing a skin temperature in proximity to the at least one US transducer generating first skin temperature data based thereon;
   analyzing said first skin temperature data and said electrical parameter to determine a subcutaneous temperature; and
   analyzing a temporal profile of said subcutaneous temperature and controllably operating the at least one transducer based on identified temporal profile behaviors of said temporal profile of said subcutaneous temperature.
40. The method of any one of embodiments 29-39, comprising measuring tightening force of the device around the body part caused by said fixing; wherein the method comprises generating a tightening alert if the tightening force is outside of an allowed tightening force range.
41. The method of embodiment 40, wherein said measuring is performed by at least one force sensor selected from: force-sensing resistor or strain gauge.
42. The method of any one of embodiments 29-41, comprising:
   sensing acceleration of the subject and generating acceleration data based thereon;
   analyzing said acceleration data and determining if the subject is moving; and
   if a movement of the subject is determined, disabling the operation of the at least one US transducer.
43. The method of any one of embodiments 29-42, comprising:
   sensing a skin temperature in proximity to the at least one US transducer and generating second skin temperature data based thereon;
   analyzing said second temperature data to determine if the temperature of the skin exceeds an allowed temperature threshold; and
   stopping the operation of the at least one US transducer upon identification of said exceeding of said allowed temperature threshold.
44. The method of any one of embodiments 29-43, comprising:
   detecting acoustic signal from skin portion of the subject; and
   generating acoustic sensed data, said acoustic sensed data being indicative of blood flow in said blood vessel or tissue;
   wherein said sensed data comprises said acoustic sensed data.
45. The method of any one of embodiments 29-44, wherein said response to said sensed data comprises identification of a change of said at least one physiological parameter;
   wherein upon identification of said change at least one of the following is being performed: (a) said US pulse is transmitted to said at least one vessel or tissue, such that an on-demand treatment is provided; (b) at least one treatment parameter of said US pulse transmission to said at least one vessel or tissue is changed, such that an as-needed treatment is provided; (c) outputting a notification of said change; (d) any combination thereof.
46. The method of any one of embodiments 29-45, wherein said at least one sensor comprises one or more of the following: accelerometer, impedance measurement, photoplethysmography sensor, PPG sensor, pH sensor, ultrasound sensor, echocardiogram, ultrasound echo, hydrophone, temperature meter, body core temperature, heart pulse rate, pulse wave properties, glucose sensor, any sensor indicating a change in cardiac output, any sensor indicating blood pressure, any sensor indicating initiation of a dialysis session, any sensor associated with dialysis machine and any combination thereof.
47. The method of embodiment 46, wherein said sensor is adapted to sense at least one parameter selected from a group consisting of: movement of said patient, impedance, PPG signal, acoustic signal, pressure, temperature, heart rate, pulse wave properties, glucose level, blood pressure, and any combination thereof.
48. The method according to embodiments 29-47, wherein said physiological parameter or state or change thereof is selected from one or more of the following: position of the patient, engagement in physical activity, decrease in NO levels in said at least one vessel or tissue, tissue perfusion, initiation of a physical activity, change in at least one parameter associated with said physical activity, the position of the patient relative to the ground, changes in said position of the patient relative to the ground, application of at least one medical treatment to said patient, changes in application of at least one medical treatment to said patient and any combination thereof.
49. The method of any one of embodiments 29-48, wherein said operating comprises controlling at least one treatment parameter selected from a group consisting of: a phase, an operating frequency, power, intensity, operating amplitude, timing, duration, orientation, alignment and any combination thereof of said ultrasonic energy.
50. The method of any one of embodiments 29-49, wherein, upon change of said at least one physiological parameter or state, at least one notification is sent to said patient or any care giver thereof.
51. The method of any one of embodiments 29-50, wherein said operating results in a physiologic effect selected from one or more of the following: vasodilation; an increase in local nitric oxide; enhanced nitric oxide release from the vascular endothelium; prolonged local nitric oxide effects; an alteration in the function of erythrocytes; a modification in oxygen release from hemoglobin, blood temperature increase; modification in pH of blood; modulation in the immune response of blood leucocytes; modulation of the coagulation and/or thrombocyte function;
modification in the function of heme catalyst enzymes in the blood, improved bioavailability of medication, improved efficiency of a hemodialysis session artery dilation, increased blood perfusion and combinations thereof.
52. The method of any one of embodiments 29-51, wherein said non-invasively applying ultrasonic (US) pulses on or towards a blood vessel or tissue of a subject is for treatment of one or more of: pulmonary artery denervation, pulmonary hypertension, ischemic tissues, PAD, CLI, pulmonary artery hypertension, arteriovenous fistula (AVF) maturation, Raynaud disease, severe asthma patients, improve blood flow to the brain during stroke, increase blood flow to the penis to maintain an erection, enhancement of bioavailability of medications, enhancement of local chemotherapy absorption into a solid tumor by enhancing flow of specific arteries feeding the tumor and any combination thereof.
53. The method of embodiment 52, wherein said ischemic tissue is selected from a group consisting of the upper limbs and lower limbs, arms, legs and any combination thereof.
54. The method of any one of embodiments 29-53, wherein said operating results in an increase of blood flow.
55. The method of any one of embodiments 29-54, wherein said at least one US transducer comprises an array of US transducers, wherein the method further comprises controllably operating the array of US transducers for steering the US pulse to a desired direction.
56. The method of embodiment 55, wherein the array of US transducers operates as a phased array for said steering.
57. The method of embodiment 55 or 56, comprising executing a periodical control loop that comprises controllably operating the array of US transducers with varying sets of operation parameters and analyzing the sensed data associated with time windows of operation of each set of operation parameters and identifying the optimal set of operation parameters that yield the optimal result of the at least one physiological parameter; wherein upon identification of said optimal set of operation parameters, the method further comprises controllably operating the plurality of US transducers in said optimal set of operation parameters until the next periodical control loop.
58. The system of embodiment 57, wherein the varying sets of operation parameters comprise varying intensity, phase or duty cycle of each US transducer of the array of transducers.
59. A system for treatment of a patient's blood vessel, comprising:
   a blood vessel sleeve configured to be fitted around the outside of at least a portion of a blood vessel, the sleeve comprising at least one ultrasonic (US) transducer configured to provide ultrasonic energy to said at least a portion of a blood vessel, and a first sensor configured to monitor at least one physiological parameter of the patient and generate sensed data based thereon; and
   a remote controller disposed external to the patient, the remote controller being data communicable with said at least one ultrasonic transducer and said first sensor for controllably operating the at least one ultrasonic transducer and receiving sensed data from the sensor.
60. The system of embodiment 59, comprising an energy transmitting unit configured to be placed externally to the patient and an energy receiving unit coupled to or integral with the blood vessel sleeve; wherein the energy transmitting unit is configured to transmit energy externally to the patient towards the energy receiving unit for energizing the US transducer.
61. The system of embodiment 60, wherein the remote controller comprises said energy transmitting unit.
62. The system of embodiment 60 or 61, wherein the energy receiving unit is a first inductor and the energy transmitting unit is a second inductor.
63. The system of any one of embodiments 59-62, wherein the energy receiving unit is coupled to the blood vessel sleeve by a pig-tail connection.
64. The system of any one of embodiments 59-63, wherein the blood vessel sleeve comprises a light unit configured to emit light towards said blood vessel.
65. A method for treatment of a patient's blood vessel, comprising:
   fitting a blood vessel sleeve around the outside of at least a portion of a blood vessel, the sleeve comprising at least one ultrasonic (US) transducer configured to provide ultrasonic energy to said at least a portion of a blood vessel, and a first sensor configured to monitor at least one physiological parameter of the patient and generate sensed data based thereon;
   controllably operating the at least one ultrasonic transducer and receiving sensed data from the sensor.
66. The method of embodiment 65, wherein said blood vessel is a bypass blood vessel bypassing an artery narrowing portion.
67. The method of embodiment 65 or 66, comprising emitting light from said sleeve towards said blood vessel.

### BRIEF DESCRIPTION OF THE FIGURES

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements.

The figures are listed below.

FIG. 1 schematically illustrates a wearable device that applies ultrasonic energy towards an artery, according to an aspect of the present disclosure.

FIG. 2 schematically illustrates another wearable device that applies ultrasonic energy towards an artery, according to an aspect of the present disclosure.

FIG. 3a shows an exemplary pulsed mode ultrasound generation by the wearable device, according to an aspect of the present disclosure.

FIG. 3b shows an exemplary operation protocol of the device, according to an aspect of the present disclosure.

FIG. 4a illustrates an exemplary two center frequencies piezoelectric transducer, according to an aspect of the present disclosure.

FIG. 4b shows an exemplary geometric arrangement of an array of piezoelectric elements of the piezoelectric transducer.

FIG. 5 illustrates an exemplary on-skin piezoelectric transducer utilized as an acoustic sensor, according to an aspect of the present disclosure.

FIG. 6 shows exemplary analysis of ECG and pulse wave signal analysis, according to an aspect of the present disclosure.

FIG. 7 shows an exemplary piezoelectric electrical terminals charge-discharge current waveform, according to an aspect of the present disclosure.

FIG. 8 illustrates an exemplary waveform generated by the piezoelectric transducer and its accompanying circuitry in response to leg movements, according to an aspect of the present disclosure.

FIG. 9 is a schematic illustration of a system, according to an aspect of the present disclosure.

FIGS. 10-11 are schematic illustrations of different solutions for providing energy to the system, according to an aspect of the present disclosure.

FIG. 12 is a schematic illustration of a cross section of a non-limiting example of a system, according to an aspect of the present disclosure.

### DETAILED DESCRIPTION OF SPECIFIC EMBODIMENTS OF THE INVENTION

As used herein with reference to quantity or value, the term “about” means “within ±10 % of”. The terms “comprises”, “comprising”, “includes”, “including”, “has”, “having” and their conjugates mean “including but not limited to”.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The present invention discloses a device that comprises at least one transducer (e.g., piezoelectric) which generates either ultrasonic energy or vibrational energy to mimic application of shear forces on the endothelium cell to induce NO production and release.

The device would be placed in proximity to ischemic tissues (namely, the upper limbs and lower limbs, i.e., the arms and legs).

Activation of the device to provide vibrations or ultrasonic energy could primally affect local NO release from endothelium cells. Additionally, such device could induce ATP release. It should be noted that, it is within the scope of the present invention to provide the device constructed and arranged to cause any other physiologic effect, for example: blood temperature increase; vasodilation; prolong local nitric oxide effects; alteration in the function of erythrocytes; modification in oxygen release from hemoglobin; modification in pH of blood; modulation in the immune response of blood leucocytes; modulation of the coagulation and/or thrombocyte function; modification in the function of heme catalyst enzymes in the blood, improved bioavailability of medication, improved efficiency of a hemodialysis session and combinations thereof.

According to another embodiment, the device would be activated from outside the patient's body. Thus, according to this embodiment, a remote controller that comprise an electronic communication device external to the patient, which is in communication with the device, adapted to activate the same and can be used by a ‘healthcare’ provider to program and control the source of energy and thus, the treatment protocol delivered to the patient (e.g., increase/decrease amount, time, level of energy, source of energy etc.).

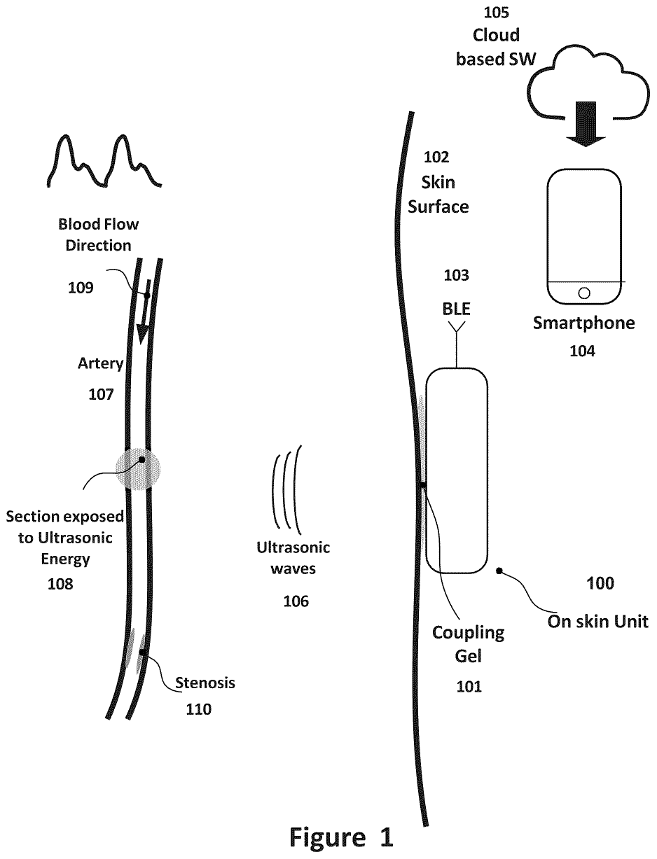

Reference is now made to FIG. 1, illustrating a device 100 placed on a skin surface 102 of the patient against an artery 107. A coupling gel 101 may be used between the device radiating surface and the skin surface. Blood flow direction is indicated by 109. The artery may have a stenosis 110. Upon activation of the transducer, mechanical vibrations 106 are induced on a section 108 of the blood vessel 107. One skilled in the art would appreciate that the device 100 may utilize any number of transducers. The device 100 may communicate with an external unit 104, such as a smartphone, via a wireless link, such as Bluetooth 103. The device may communicate with a cloud-based program 105 directly or through the external unit 104.

Furthermore, by providing many transducers, the position and orientation of each transducer could be controlled. Yet more, control of the position and orientation of each of the transducers provides focusing capability on the desired blood.

According to another embodiment, as there are multiple transducers, each could be operated in a different center frequency in the range (e.g., a first range of 10 kHz to about 10 MHz and a second range of 100 kHz to about 10 MHz. According to another embodiment, all transducers operate in the same center frequency range.

The device may control at least one parameter, such as: the amplitude (and therefore the intensity or power) of the acoustic wave transmitted by the transducer, the timing (e.g., start and period), a refractory period (i.e., do not start a new session before at least X minutes have passed from the last session), directionality of the signal (one transmitter can stimulate more than one blood vessels located slightly away from each other and the phased array can point the stimulation to a different one each time) and any combination thereof. In other embodiments, if the device includes more than one transducer, such as a piezoelectric-based transducer,, it may also control a phase component of the drive signals to respective transducer elements of the transducer device, e.g., to control a shape or size of a focal zone generated by the transducer elements and/or to move the focal zone to a desired location. For example, the controller may control the phase shift of the drive signals to adjust a focal distance (i.e., the distance from the face of the transducer to the center of the focal zone). In further embodiments, the device may operate the transducer for a predetermined duration. Alternatively, or additionally, the controller of the device can be configured to automatically turn off the transducer when a usage of the transducer exceeds the predetermined time.

As disclosed above, according to one embodiment, the device can be in communication with at least one sensor, which may include, for example, an accelerometer, impedance sensor, photoplethysmography sensor, PPG sensor, pH sensor, ultrasound sensor, hydrophone, temperature meter, body core temperature, heart pulse rate, pulse wave properties, glucose sensor, manual activation, any sensor indicating a change in cardiac output, any sensor indicating blood pressure, any sensor indicating initiation of a dialysis session, any sensor associated with dialysis machine and any combination thereof. In such an embodiment the device is an ‘on-demand’ device. In other words, the ‘on demand’ device will be activated only when needed (‘on-demand’ basis). According to this embodiment, the sensor(s) monitors physiological state(s) of the patient such that, upon change thereof, the controller signals the transducer to emit acoustic signal(s) (or to amend one of the acoustic signal's parameters).

As specified, each of the sensors is adapted to monitor the physiological state of the patient and treatment is provided accordingly. For example, an accelerometer will be used to indicate if the position of the patient changes (e.g., when the patient lies down, stands, walks, etc.). Upon sensing of a change (e.g., when the patient starts to walk) the device is activated. In other words, the device is activated ‘on demand’ (when the patient engages in physical activity, e.g., walking) to induce NO release (and thus, increase oxidation levels/tissue perfusion in the tissue and decrease any pain associated therewith).

According to one embodiment, the treated target tissue is one that has been affected by a peripheral arterial disease (and is located within the upper or lower limbs of the patient). In other embodiments, the target tissue can be associated with other diseases or medical conditions (such as pain due to exercising), and can be located at other parts of the patient's body.

Once the device is positioned in its predetermined location, the transducer (upon a signal from the device controller) delivers ultrasound energy to the target tissue. The transducer may emit acoustic energy in a continuous manner, or alternatively, in pulses. In some embodiments, the device controller may also control, for example, the phase, an operating center frequency, temperature of the tissue (to ensure the tissue is not overheated) and/or an operating amplitude of the transducer.

According to another embodiment, the device may include a tissue electrical impedance sensor.

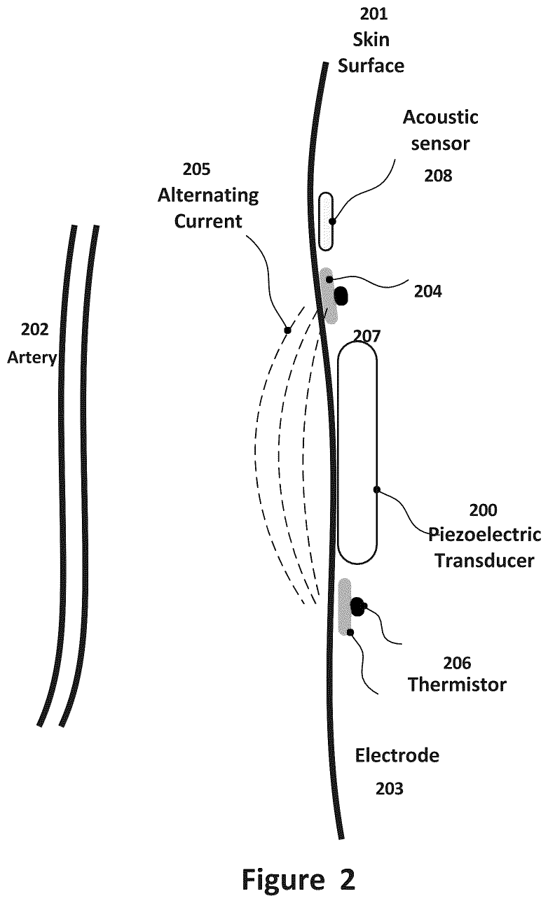

Reference is now made to FIG. 2, which illustrates an ultrasonic transducer 200 (e.g., a piezoelectric transducer) attached to the skin surface 201 against an artery 202. Electrically conducting electrodes 203, 204 may be employed and attached to the skin surface 201. These electrodes may be excited with a high center frequency alternating signal (e.g. 100 kHz). the excitation causes alternating current 205 to flow through the subcutaneous tissue. The induced alternating current may be measured, and the equivalent electrical impedance can be calculated. If the device is not correctly attached to the skin surface, the electrical impedance is changed, and the device controller may consequently stop the ultrasound radiation, in order not to cause overheating of the skin surface.

According to another embodiment, the electrode excitation center frequency may be swept in order to more accurately estimate the temperature of the subcutaneous tissue exposed to the ultrasound radiation.

According to another embodiment, thermal sensors 206, 207 such as thermistors, may be employed to measure the tissue surface temperature. The thermal sensors may be attached to the skin surface, or attached to the electrodes.

In another embodiment, the electrodes may be used also as ECG electrodes.

In another embodiment, at least one acoustic sensor 208 may be employed for sensing the blood pulse wave while it flows through the artery 202. The pulse wave measurement by the acoustic sensor may indicate the blood flow profile in the artery and therefore the effect of the insonation can be evaluated. Optionally, the insonation parameters may be adjusted according to the blood flow profile that is determined by the measurement of the acoustic sensor.

It is to be noted that many types of sensors can be included in the system of FIG. 2 other than acoustic sensor(s) and thermal sensor(s). For example, the system may include ECG sensor(s).

The processing circuitry that controls the operation of the system of FIG. 2, namely the transducer and the sensors, may be configured to execute at least one of the following: (1) a check US pulse to determine the contact quality between the transducer and the skin of the subject by analyzing the detected echoes of the check US pulse; (2) an electric measurement between two electrodes disposed on both sides of the transducer to determine an electric parameter indicative of the contact quality between the transducer and the skin of the subject; a periodical steering control loop, in which the angle of the transducer with respect to the skin of the subject and/or the angle of the beam of the US pulse are varied and the optimal steering state is selected according to a measurement of the physiological parameter, e.g. according to the pulse wave that is measured by the acoustic sensor. It is to be noted that (1), (2) and (3) can either be executed independently or simultaneously. For example, the execution of the check US pulse and the electric measurement may provide higher confidence level for the quality of the contact of the transducer with the skin.

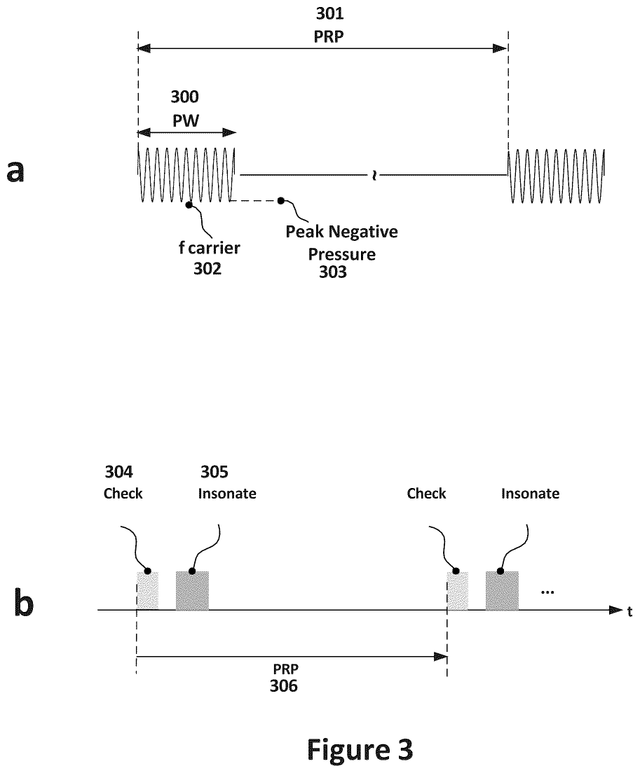

Reference is now made to FIG. 3a, which shows an exemplary ultrasonic wave that the device radiates toward the artery. The ultrasonic wave may be in the form of a pulse 300, with a pulse repetition period PRP 301. The pulse may contain several pressure cycles to thousands of cycles, with a constant center frequency 302 and peak negative pressure under the cavitation threshold 303. In another embodiment, the center frequency and/or the peak negative pressure amplitude may be altered from pulse to pulse.

In another embodiment, open-loop or closed-loop transducer resonance frequency tracking may be utilized.

Referring to FIG. 3b, the operation protocol may be based on a pulse repetition period 306 that includes a first sensing phase 304 followed by an insonation pulse 305. The sensing phase allows the device controller to verify correct placement and skin attachment of the device and tissue temperature below a safety threshold level before it excites the piezoelectric transducer.

According to another embodiment, the piezoelectric transducer may generate ultrasound in more than one center frequency. Ultrasound wave containing more than one center frequency is known to augment the effect in the target: Schoellhammer C M, Polat B E, Mendenhall J, Maa R, Jones B, Hart D P, Langer R, Blankschtein D. Rapid skin permeabilization by the simultaneous application of dual-frequency, high-intensity ultrasound. J Control Release. 2012 Oct. 28; 163(2):154-60.

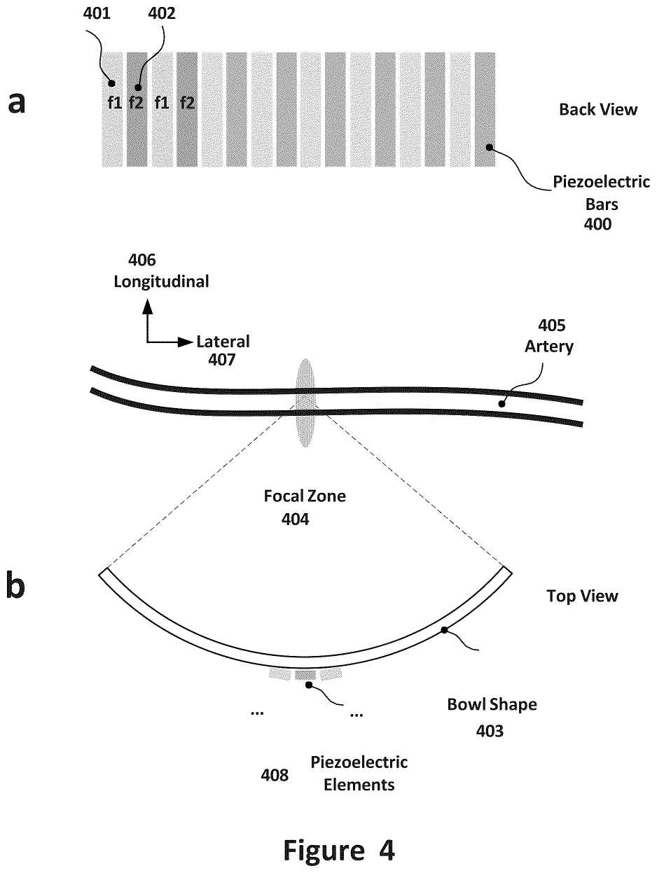

Referring to FIG. 4a, a possible realization of piezoelectric transducer is shown, where the transducer is based on an array of piezoelectric elements such as bars 400. The piezoelectric elements may be arranged such that each element may alternately have a different resonant frequency, such as f1 401, f2 402, where f1 and f2 are the vibration resonance of the piezoelectric elements.

Referring to FIG. 4b, the piezoelectric array may be arranged in a concave/bowl 403 shape or any other geometrical shape that can geometrically focus the energy in a longitudinal direction 406 to a focal zone 404 over the artery 405 to increase the shear force on the artery wall in the lateral direction 407.

According to another embodiment, pulse wave characteristics and pulse wave velocity can be measured to indicate artery dilation and increased blood perfusion.

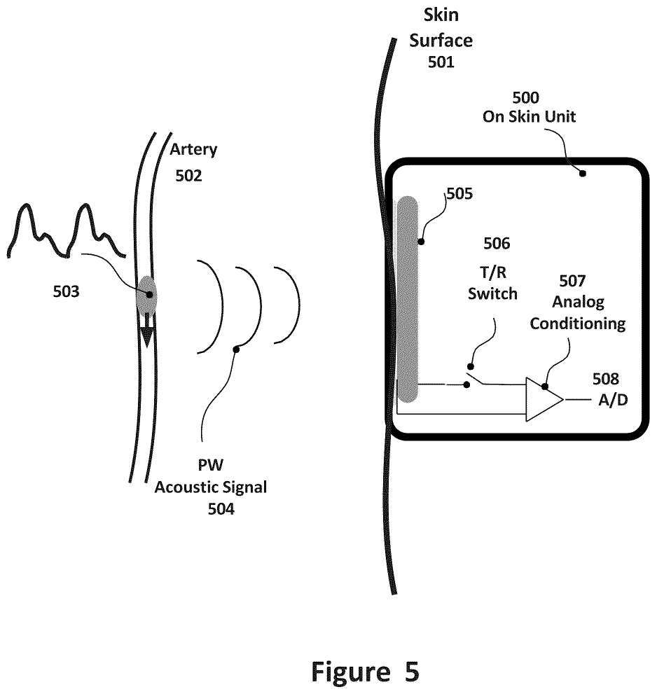

Reference is now made to FIG. 5, illustrating such acoustic sensing of pulse wave characteristics. As can be seen in FIG. 5, blood pulse incident and reflected waves 503 flowing in the artery 502 results in vibrations 504 that propagates also toward the skin surface 501. The on-skin unit 500 contains an acoustic sensor 505 that senses the vibration 504 and converts it to an electric signal at its electrical terminal. In another embodiment, the ultrasound generating transducer may be employed also as an acoustic sensor 505. A protection switch 506 may be employed in order to protect the electronic conditioning circuitry 507 from the high voltage pulses used to excite the ultrasonic transducer. The output signal of the electronic conditioning circuitry 508 may be digitized by the device controller. Analysis of the signal can provide indication for dilation and increased tissue perfusion and for optimized position and/or orientation of the transducer relative to the blood vessel. Thus, this analysis can facilitate alignment of the positioning of the device transducer relatively to the vessel or tissue containing flowing blood.

According to another embodiment, the alignment is indicated (either to the patient or the caregiver) by at least one indication means, such as audio means, visual means, tactile means and any combination thereof.

According to another embodiment, the analysis of the pulse wave characteristics indicates if the desired physiologic effect (vessel dilation and increased blood perfusion) is indeed achieved.

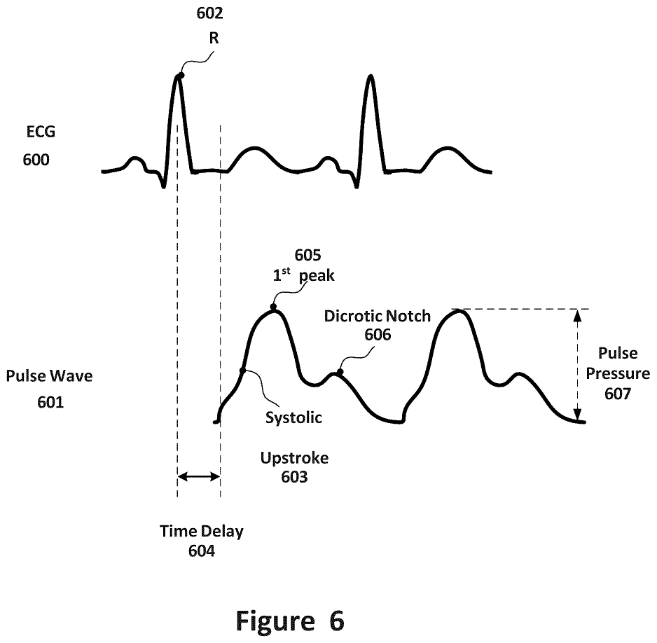

Referring to FIG. 6, shown are exemplary ECG signal 600 and pulse wave signal 601. A time delay 604 exists between the R part 602 of the ECG signal 600 and the systolic upstroke start 603 of the pulse wave signal 601. The delay depends on the downstream blood vessels stiffness (Kwon Y, Jacobs D R Jr, Lutsey P L, Brumback L, Chirinos J A, Mariani S, Redline S, Duprez D A. “Sleep disordered breathing and ECG R-wave to radial artery pulse delay, The Multi-Ethnic Study of Atherosclerosis”. Sleep Med. 2018 August; 48:172-179. doi: 10.1016/j.sleep.2018.05.005. Epub 2018 May 21). Shown also are the peak systolic 605, the dicrotic notch 606 and the pulse pressure 607. All contain valuable information on arterial stiffness and downstream blood flow resistance. Sensing the pulse wave waveform enables long term tracking of clinical conditions, such as PAD progress, and also titrate the ultrasound dose accordingly in a closed-loop form.

According to one embodiment, the on-skin unit will acoustically sense at least one pulse wave characteristic, reflected from a blood artery.

It is within the scope of the present disclosure where the analysis of the pulse wave characteristic(s) facilitate alignment of the position of at least one of a plurality of devices relatively to the treated vessel or tissue containing flowing blood. It should also be appreciated that it is within the scope of the present disclosure where the analysis of the pulse wave characteristic(s) indicates if the desired physiologic effect has been induced (e.g., artery dilation and increased blood perfusion).

It is crucial to maintain a good coupling attachment of the radiating surface of the transducer to the tissue surface. Since the patient may be active, his/her movements may impact the coupling. If the coupling is not good enough, the ultrasound energy may not be transferred to the skin and tissue, and most of the electrical energy driven to the transducer by the driver will be converted into heat, which will rapidly climb to a level that may cause burns and damage to the tissue. In order to verify that the transducer-skin coupling is proper, and remains proper, the equivalent electrical impedance of the transducer at its working vibration resonance frequency may be sensed. The resistive part of the electrical impedance depends on the mechanical load as seen from the transducer's radiating surface.

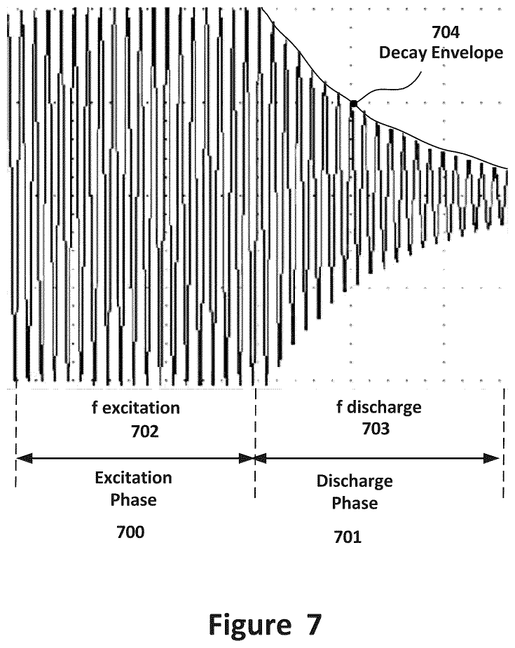

Referring to FIG. 7, in order to sense the impedance, the driver may excite the transducer with low voltage ac signal, such as 1 volt, in a frequency ‘f excitation’ 702 that is close enough to the operating vibration frequency of the transducer. Excitation phase 700 may last for several tens of excitation cycles, and then the driver stops the excitation phase and transitions to the discharge phase, where it forces a short circuit on the electrical terminals of the transducer. During the short circuit of the discharge phase 701, the transducer's vibration energy is discharged, and a discharge current flows through its electrical terminals that are held short circuited. The discharge current frequency ‘f discharge’ 703 is in its natural vibration frequency. The current decay envelope 704 depends on the coupling quality of the transducer to the skin.

The piezoelectric nature of the ultrasonic transducer of the device enables to use the transducer as a patient motion sensor while it is not excited.

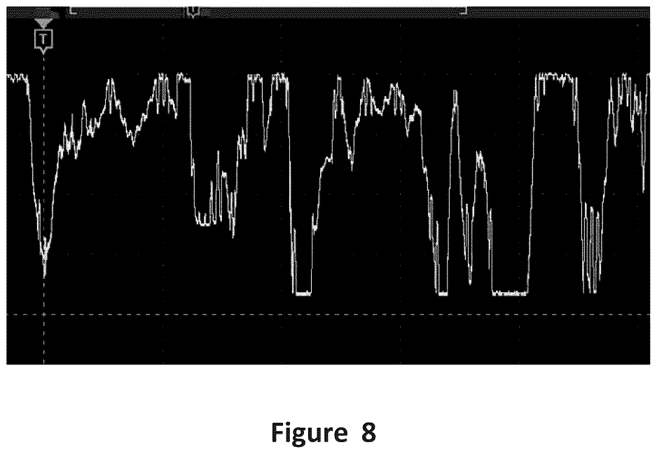

Referring to FIG. 8, shown is an exemplary electrical voltage signal as measured at the electrical terminals of the ultrasonic transducer while the patient is moving (e.g., in response to leg movements).

According to another embodiment, the device components (the battery, the electronic circuitry, etc.) may be distributed into several subunits. The distribution into several subunits enables to distribute the weight more evenly and minimize the height of each component above the skin, allowing for a low profile device.

According to another embodiment, the connection between the various components of the device is achieved with a flexible inflatable sleeve.

According to another embodiment, the device (on-skin unit) 100 of FIG. 1 may be in communication with an external sensor, such as ECG sensor. The external sensor may be a wearable sensor. The external ECG sensor sends a synchronization pulse indicating the R part of the signal, for instance. According to one embodiment, the wearable ECG sensor may be used continuously. Alternatively, the ECG sensor may be used discreetly (i.e., from time to time).

It should be noted that according to this embodiment, during a treatment session, the energy intensity or dosage delivered by the transducer at the tissue is kept below a prescribed threshold (e.g., by using appropriate driving scheme and/or by selecting appropriate operation parameters, such as an operating center frequency, an operating amplitude, etc.), thereby protecting the tissue from being damaged by the acoustic energy.

In other embodiments, the transducer can be moved relative to the patient. In such embodiments, the position of at least one of the transducers can be optimized relatively to the treated tissue or blood vessel.

According to another embodiment, multiple on-skin devices are utilized. Such an embodiment provides multiple points of treatment along the blood vessel (resulting in a large range of effect) and analysis of the pulse wave characteristics may be used to optimize treatment parameters and positioning (e.g., alignment) of at least one of the devices relative to the blood vessel or tissue.

According to another embodiment, the piezoelectric transducer of the on-skin ultrasonic device may be floating within a partially soft material chamber filled with electrically non-conducting fluid, such as silicone oil. The floating transducer is therefore more protected from mechanical impacts.

According to another embodiment, the piezoelectric transducer chamber is inflatable in order to maintain enough acoustic coupling of the acoustic/ultrasound wave to the skin.

According to another embodiment, the floating piezoelectric transducer is tilted in order to improve its alignment and orientation toward a blood vessel. In another embodiment, the piezoelectric transducer may be moved in the chamber in order to change its position relative to a blood vessel. Alignment may be achieved by: (a) electromagnetic mechanism, such as, but not limited to, magnetic bearings used in flywheel energy storage or magnetic levitation (b) piezoelectric mechanism, (c) electrostatic mechanism, and any combination thereof.

According to another embodiment, the device is provided with a gel holder. The holder has openings in the vicinity of the ultrasound emitting or receiving surface. The gel contained in the gel holder is automatically or semi-automatically dispensed from the gel holder through the openings in the ultrasound radiating surface, in order to maintain proper ultrasound coupling between the device radiating surface and the skin. According to another embodiment, the gel holder may be made of flexible material, and the patient may press the gel holder in order to cause gel to be released from the holder through the openings.

According to another embodiment, the device may be embedded in a stretchable sleeve. The sleeve may be stretched automatically before insonation, to ensure good ultrasound coupling to the skin.

In another embodiment, the device may be embedded in an inflatable sleeve. In another embodiment, only the sub-unit containing the piezoelectric transducer is inflated. The controller of the device may inflate the sleeve automatically before insonation in order to ensure proper ultrasound coupling to the skin. In another embodiment, the controller inflates the sleeve to a predetermined pressure level. The pressure may be sensed using a pressure sensor, force sensor, or by using the device piezoelectric transducer as a coupling sensing mechanism using charge-discharge method, as described, for example, in FIG. 7.

It is to be noted that the system can be arranged in any desired arrangement and its components may be distributed along the limb of the subjected according to any desired design. There is no limitation for the distribution of the components of the system.

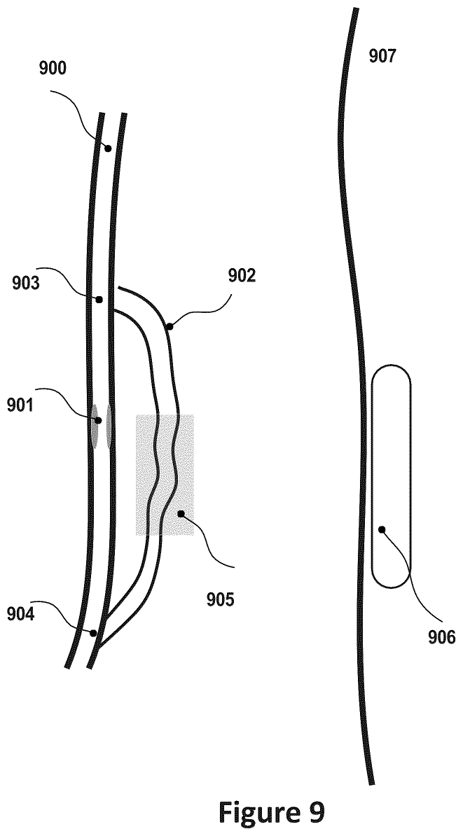

Reference is now being made to FIG. 9, which is a schematic illustration of a non-limiting example of the system according to an aspect of the present disclosure. The system comprises a blood vessel sleeve 905 being fitted over an external portion of a bypass blood vessel 902 (e.g., arteriovenous fistula (AVF)). In the drawing, an artery 900 has a narrowing section 901 which limits blood flow. A bypass blood vessel 902 is connected to the artery 900 at the connection positions 903 and 904 to enable blood flow that bypasses the artery narrowing section 901. The device 905 comprises ultrasonic transducers that applies ultrasonic energy to the bypass blood vessel. The ultrasonic energy dose is given in a low duty cycle, e.g., a couple of times a day, each time for a short duration, for example several minutes. the operation of the device 905 is controlled by an external controller 906 that is coupled to the skin surface 907. The blood vessel sleeve 905 may give external mechanical support to the bypass vessel by limiting its swelling potential. It may also induce shear force on the artery endothelial cells and on the red blood cells flowing in the bypass blood vessel, and consequently cause the release of nitric oxide (NO) that may cause vasodilation and increased blood flow. The blood vessel sleeve 905 may also minimize the abnormal proliferation of vascular smooth muscle cells (VSMCs) that is known to be a key event in the development of blood vessel restenosis. The blood vessel sleeve 905 may comprise a light source, such as a laser diode, to emit light onto the blood vessel to promote the arteriovenous fistula (AVF) maturation. The light emitted by the light source may be in the IR spectrum, or more specifically in the far-IR spectrum.

The blood vessel sleeve 905 may include a blood flow measurement sensor and/or a temperature sensor for sensing physiological parameters indicating the blood flow and the state of the bypass blood vessel.

The blood vessel sleeve 905 may include a heat generating source, such as resistive, optical energy source, or ultrasonic generating source. The heat source couples heat to a blood vessel section. The heat source may be activated in pulsed mode in order to facilitate blood flow velocity measurement through a blood vessel. This may also promote AVF maturation or increase of blood flow in the blood vessel.

The blood vessel sleeve 905 may comprise its own battery to supply it energy or the energy can be supplied by the external controller 906 through wireless transmission of energy.

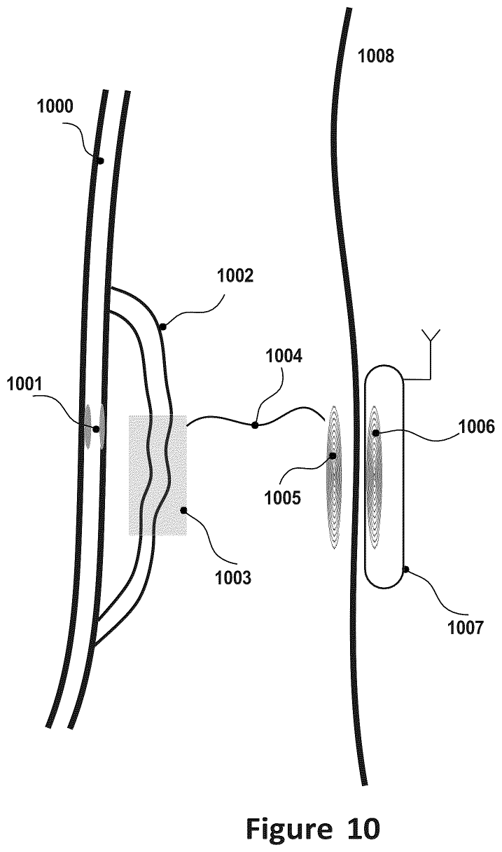

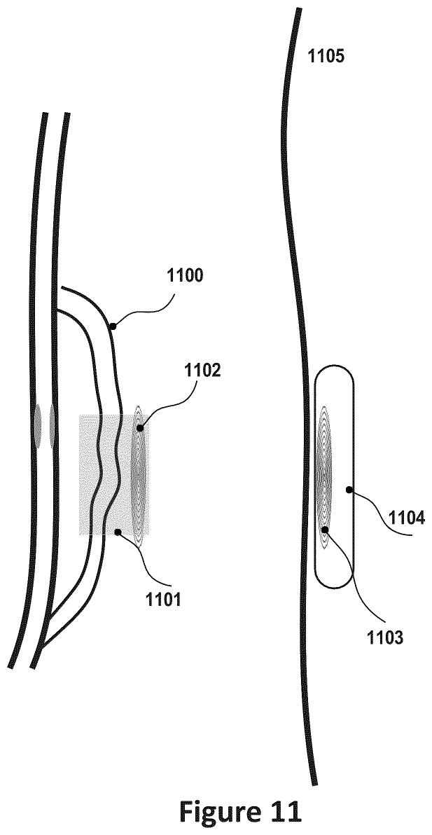

FIGS. 10-11 show different solutions for providing energy to the blood vessel sleeve.

FIG. 10 shows a blood vessel sleeve 1003 located over a bypass blood vessel 1002 to bypass a narrowing section 1001. The sleeve 1003 comprises an electrical wiring in the form of a pig-tail 1004 that is coupled to an inductor 1005, for instance in the shape of a flat spiral inductor. The inductor 1005 may be placed subcutaneously. An on-skin unit 1007 coupled to the external skin surface 1008 may incorporate an inductor 1006 to be placed against the subcutaneous inductor 1005 to form a wireless power and data transfer inductive link.

In another embodiment, the wireless link at the end of the pigtail 1004 may be utilized as an ultrasonic wireless link, or a capacitive wireless link or a combination thereof.

FIG. 11 illustrates another embodiment of a bypass device wireless power and data transfer. In the figure, a blood vessel sleeve 1101 is placed around a bypass blood vessel 1100. An inductor 1102 is placed inside the sleeve 1101, form a power and data wireless link with an external antenna 1103 that is comprised inside an external device 1104. The external device 1104 may be coupled to the external skin surface 1105.

In another embodiment, the sleeve 1101 may incorporate an ultrasonic transducer such as a flat disc shaped piezoelectric element, to form a wireless power and data transfer with an external on skin ultrasonic transducer.

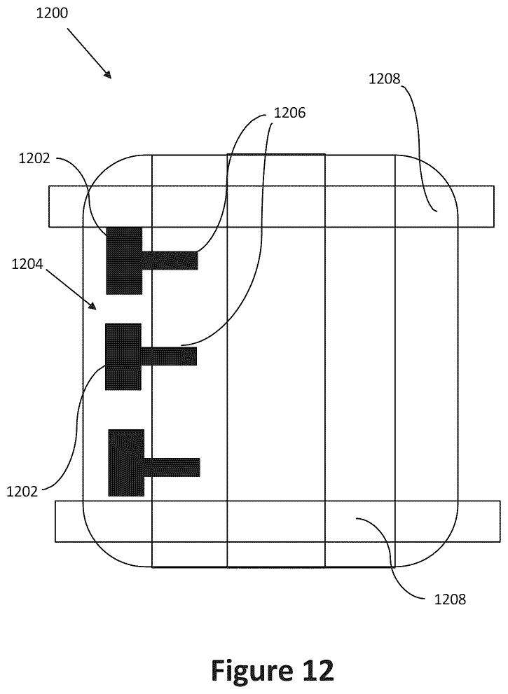

Reference is now made to FIG. 12, which is a schematic illustration of a cross section of the system for non-invasively applying ultrasonic pulses towards a blood vessel or a tissue of a subject. The system 1200 comprises a plurality of US transducers 1202, though it should be understood that the system may only include one transducer. The transducers 1202 are disposed in a chamber 1204 filled with liquid that is suitable for serving as an acoustic couplant. The transducers 1202 are steerable by steering elements 1206 that may be operated mechanically, electrically, magnetically or by any other known techniques. The system 1200 further comprises fixation assembly for fixing/securing the transducers to a body part of the subject (e.g., to a subject's limb). In one embodiments, the fixation assembly is constituted by one or more straps 1208. The straps 1028 may be attached to the subject's body (e.g., to a limb) using buckles, a hooks and loops mechanism (e.g., Velcro®), and the like. By employing this configuration, the transducers 1202 can be easily steered towards a desired location which is identified as the location of the blood vessel and the fixation of the system to the limb of the subject ensures that there is a good acoustic coupling between the transducers 1202 and the subject's body so as to allow the US pulses to be transmitted to the blood vessel or tissue. The steering state of the transducers can be optimized by the processing circuitry that controls the operation of the steering elements 1206. For example, the angle of the steering elements 1206 can be optimized by execution of a steering control loop. During the control loop, varying combinations of angles may be used for insonation. At least one physiological parameter is monitored during the control loop, for example a parameter that is indicative of the blood flow in a blood vessel. By the end of the control loop, the processing circuitry analyzes the at least one physiological parameter in a plurality of the time windows, each time window is associated with a specific combination of angles of the steering elements 1206. The combination of angles that derives the optimal parameter for the at least one physiological parameter is selected for the next period of insonation. It should be noted that the disclosed device could be used for the treatment of various clinical conditions, such as (the list below includes several non-limiting examples):
CLI/PAD—improved healing for post-revascularization patients. In such applications, the device will be activated following revascularization either constantly or intermittently.
Arteriovenous fistula (AVF) maturation for patients that have end-stage renal disease (ESRD). The disclosed device and method can be used to increase the success rate of the fistula and, further, to maintain the patency of the fistula for a longer period of time. It can also be effective in long-term monitoring of the blood flow through the fistula.
Improve PAD patients' wound healing, including saving legs from amputation.
Local and chronic therapy for pulmonary artery hypertension.
Treating severe asthma patients (severe patients which do not respond to standard asthma inhalers) by affecting the bronchial arteries. It should be noted that in such applications, any parameters associated with breathing or vocal which can provide indication of an asthma attack can trigger the activation of the device to alleviate the bronchial spasm.
Improve blood flow to the brain during stroke by generating NO in the carotid artery (using an external ultrasound unit).
“Local Viagra®” increase blood flow to the penis to maintain an erection without the systemic side effects of sildenafil (the Viagra® pill).
Improved bioavailability of medication—it should be noted that for this application, the time of medication administration (oral, IV or pump activation) can be synchronized to the activation of the ultrasound device to enhance local absorption of the medication at the target organ and the location of the device would then be in the vicinity of arteries feeding the target organ. For example, chemotherapy flow into a solid tumor could be enhanced during chemotherapeutic sessions.
Raynaud disease—by using the solution of the present invention, the feeding artery of the hand can be insonated to increase the blood flow to the fingers.

Although certain embodiments have been illustrated and described herein, it will be appreciated by those of ordinary skill in the art that a wide variety of alternative or equivalent embodiments or implementations, calculated to achieve the same or similar purposes, may be substituted for the embodiments illustrated and described herein without departing from the scope of the present invention. Those of skill in the art will readily appreciate that embodiments in accordance with the present invention may be implemented in a very wide variety of ways. This application is intended to cover any and all adaptations and/or variations of the embodiments discussed herein.

The terms and expressions which have been employed in the foregoing specification are used therein as terms of description and not of limitation, and there is no intention, in the use of such terms and expressions, to exclude equivalents of the features shown and/or described or portions thereof, it being recognized that the scope of the invention is defined and limited only by the claims that follow.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

1-32. (canceled)

33. A non-invasive system for applying ultrasonic (US) pulses on or towards a blood vessel or tissue that contains flowing blood of a subject, the system comprising:
at least one US transducer configured to transmit ultrasonic pulses;
a fixation assembly configured for fixing or securing said at least one US transducer to a body part of the subject that comprises said blood vessel or tissue;
at least one sensor configured to monitor at least one physiological parameter or state of the subject and generate sensed data based thereon; and
at least one processing circuitry configured to be in data communication with said at least one US transducer and with said at least one sensor and configured to receive said sensed data and controllably operate the at least one US transducer in response to said sensed data to transmit US pulse towards said blood vessel or tissue.

34. The system of claim 33, comprising a steering arrangement configured to controllably steer said at least one US transducer towards said blood vessel or tissue.

35. The system of claim 34, wherein said steering arrangement comprises steering elements and a chamber filled with flowable material, wherein at least a portion of said at least one US transducer is disposed within said chamber, wherein said steering elements are configured to controllably move said at least one US transducer to thereby controllably steer said at least one US transducer.

36. The system of claim 33, wherein said at least one processing circuitry is configured to:
control the at least one US transducer to apply a check US pulse and detect the reflected echoes resulting from said check pulse;
analyze said detected reflected echoes to determine whether they satisfy a predetermined condition to thereby determine whether the at least one US transducer is in a suitable contact with a skin portion of the subject; and
if said detected reflected echoes do not satisfy said predetermined condition, generate an output alert.

37. The system of claim 33, comprising first and second electrodes, wherein the at least one US transducer is disposed between said first and second electrodes and wherein the at least one processing circuitry is configured to:
apply an electric current from the first electrode to the second electrode so as to measure an electrical parameter selected from impedance, current and voltage, the electrical parameter being indicative of a quality of contact between skin portion of the subject and the at least one US transducer; and
if the quality of contact is below a selected threshold, generate an output alert.

38. The system of claim 37, wherein said at least one sensor comprises a first temperature sensor configured to sense a skin temperature in proximity to the at least one US transducer and to generate first skin temperature data based thereon; and
wherein the at least one processing circuitry is configured to:
analyze said first skin temperature data and said electrical parameter to determine a subcutaneous temperature;
analyze a temporal profile of said subcutaneous temperature; and
controllably operate the at least one transducer based on identified temporal profile behaviors of said temporal profile of said subcutaneous temperature.

39. The system of claim 33, wherein said at least one sensor comprises at least one force sensor configured to measure the tightening force of the device around the body part caused by the fixation assembly; and wherein the at least one processing circuitry is configured to generate a tightening alert if the tightening force is outside of an allowed tightening force range.

40. The system of claim 33, wherein said at least one sensor comprises an accelerometer configured to sense acceleration of the subject and to generate acceleration data based thereon; and wherein the at least one processing circuitry is configured to:
analyze said acceleration data to determine if the subject is moving; and
if movement of the subject is determined, disable the operation of the at least one US transducer.

41. The system of claim 33, wherein said at least one sensor comprises a second temperature sensor configured to sense a skin temperature in proximity to the at least one US transducer and to generate second skin temperature data based thereon; and wherein the at least one processing circuitry is configured to analyze said second temperature data to determine if the temperature of the skin exceeds an allowed temperature threshold and to stop the operation of the at least one US transducer upon identification of said exceeding of said allowed temperature threshold.

42. The system of claim 33, wherein said at least one sensor comprises an acoustic sensor configured to detect acoustic signals from a skin portion of the subject and generate acoustic sensed data, said acoustic sensed data being indicative of blood flow in said blood vessel or tissue; and wherein said sensed data comprises said acoustic sensed data.

43. The system of claim 33, wherein said response to said sensed data comprises identification of a change of said at least one physiological parameter; and wherein upon identification of said change, at least one of the following is performed: (a) said US pulse is transmitted to said at least one vessel or tissue, such that an on-demand treatment is provided; (b) at least one treatment parameter of said US pulse transmission to said at least one vessel or tissue is changed, such that an as-needed treatment is provided; (c) outputting a notification of said change; (d) any combination thereof.

44. The system of claim 33, wherein the at least one sensor is configured to sense at least one pulse wave characteristic and determine heart rate of the subject from said at least one pulse wave characteristic; and wherein analysis of said at least one pulse wave characteristic facilitates alignment of the position of said US transducer relatively to said at least one blood vessel or tissue.

45. The system of claim 33, wherein said at least one sensor is an ultrasonic sensor configured to sense ultrasonic signals.

46. The system of claim 33, comprising an array of US transducers, wherein the at least one processing circuitry is configured to controllably operate the array of US transducers for steering the US pulse to a desired direction.

47. A method for non-invasively applying ultrasonic (US) pulses on or towards a blood vessel or tissue of a subject, comprising:
fixing at least one ultrasonic (US) transducer to a body part of the subject that comprises said blood vessel or tissue;
monitoring at least one physiological parameter and generating sensed data based thereon; and
operating said at least one US transducer in response to said sensed data to transmit a US pulse towards said blood vessel or tissue.

48. The method of claim 47, wherein said operating comprises steering said at least one US transducer towards said blood vessel or tissue; and wherein the method further comprises disposing at least a portion of said at least one US transducer in a chamber filled with flowable material and controllably moving said at least one US transducer to thereby controllably steer said at least one US transducer.

49. The method of claim 47, comprising:
executing a periodical steering control loop, comprising:
steering said at least one US transducer with varying sets of steering parameters;
analyzing the sensed data associated with time windows of operation of each set of steering parameters; and
identifying an optimal set of steering parameters that yield an optimal result of the at least one physiological parameter; and
upon identification of said optimal set of steering parameters, steering said at least one US transducer in said optimal set of steering parameters until the next periodical steering control loop.

50. The method of claim 47, comprising:
applying a check US pulse with the at least one US transducer;
detecting the reflected echoes resulting from said check pulse;
analyzing said detected reflected echoes to determine whether they satisfy a predetermined condition to thereby determine whether the at least one US transducer is in a suitable contact with a skin portion of the subject; and
if said detected reflected echoes do not satisfy said predetermined condition, generating an output alert.

51. The method of claim 47, comprising:
attaching first and second electrodes to a skin portion of the subject, wherein the at least one US transducer is disposed between said first and second electrodes;
applying electric current from the first electrode to the second electrode so as to measure an electrical parameter selected from current, impedance or voltage, wherein the electrical parameter is indicative of a quality of contact between the skin portion of the subject and the at least one US transducer; and
if the quality of contact is below a selected threshold, generating an output alert.

52. The method of claim 51, comprising:
sensing a skin temperature in proximity to the at least one US transducer and generating first skin temperature data based thereon;
analyzing said first skin temperature data and said electrical parameter to determine a subcutaneous temperature;
analyzing a temporal profile of said subcutaneous temperature; and
controllably operating the at least one transducer based on identified temporal profile behaviors of said temporal profile of said subcutaneous temperature.

53. The method of claim 47, wherein said at least one US transducer comprises an array of US transducers, wherein the method further comprises controllably operating the array of US transducers for steering the US pulse to a desired direction; wherein the array of US transducers operates as a phased array for said steering.
